(19) United States
(12) Patent Application Publication
(10) Pub. No.: US 2020/0163880 A1
(43) Pub. Date: May 28, 2020

(54) CATIONIC LIPOSOMES

(71) Applicant: DANMARKS TEKNISKE UNIVERSITET, Kgs. Lyngby (DK)

(72) Inventors: Thomas Lars ANDRESEN, Vanløse (DK); Simon Skjøde JENSEN, Brønshøj (DK); Jonas Rosager HENRIKSEN, Allerød (DK); Ladan PARHAMIFAR, Hillerød (DK); Rasmus Mikkel Münter LASSEN, København Ø (DK)

(21) Appl. No.: 16/630,200

(22) PCT Filed: Jul. 13, 2018
(86) PCT No.: PCT/EP2018/069087

§ 371 Date: Jan. 10, 2020

(30) Foreign Application Priority Data
Jul. 13, 2017 (EP) 17181122.7

(51) Int. Cl. A61K 9/127; A61K 31/522; A61K 47/69; A61K 39/39; A61P 35/00

(52) U.S. Cl. CPC A61K 9/1272; A61K 31/522; A61K 2039/55555; A61K 39/39; A61P 35/00; A61K 47/6911

(57) ABSTRACT

Disclosed herein are cationic liposomes suitable for specific delivery of immunomodulating agents to monocytes and dendritic cells. The cationic liposomes comprise phospholipids, cholesterol, cationic lipids, PEG and at least one active ingredient and have a zeta potential in the range of 13-25 mV. Further disclosed are uses of such cationic liposomes in various pharmaceutical applications.

### TECHNICAL FIELD

The present disclosure relates to cationic liposomes suitable for specific delivery of immunomodulating agents to monocytes and dendritic cells. More particularly, the present disclosure relates to cationic liposomes comprising phospholipids, cholesterol, cationic lipids, PEG and at least one active ingredient, and which have a zeta potential in the range of 13-25 mV and use of such cationic liposomes in various pharmaceutical applications.

### BACKGROUND

Liposomes are lipid vesicles composed of a lipid bilayer membrane enclosing an aqueous core. These vesicles are considered to have great potential as drug delivery systems for several reasons; i) various types of drugs can be delivered; hydrophilic drugs can be loaded into the aqueous compartment or hydrophobic drugs can be anchored in the membrane, ii) the therapeutic efficacy is enhanced by targeting specific immune cells, and iii) specific targeting to immune cells deliver the cargo drug to the intracellular compartment with reduced toxicity. As a result liposomes have been studied extensively for the past decades in an attempt to develop novel formulations to treat e.g. cancer and inflammation, but also to target specific tissues such as the brain, mitochondria, the ocular surface, and derma.

Following administration, the liposomes are often taken up by the mononuclear phagocyte system (MPS). This was considered to be one of the major drawbacks of early liposomal drug delivery systems since it results in decreased amount of drugs reaching the target site. The MPS consists of tissue macrophages, monocytes in the blood and their precursor cells in the bone marrow. Monocytes differentiate from hematopoietic stem cells in the bone marrow from where they are released into the blood. They can circulate for several days, before they as a result of pro-inflammatory, metabolic or immune stimuli leave the vasculature, migrate into the tissues and differentiate into macrophages or dendritic cells. Tissue macrophages especially those in the liver, spleen, and lymphatic system have high phagocytic activity and contribute to clearance of apoptotic cells, but also administrated liposomes. Monocytes in the blood also play an important role in elimination of pathogens and apoptotic host cells by phagocytosis.

This usually unwanted phagocytic ability of liposomes by cells of the MPS can be turned to an advantage by targeting these cells, monocytes and dendritic cells, in the blood to take up the administered liposome formulations. Monocytes are the first cells to be recruited to the site of inflammation or infection, making monocytes important components of the first line of defense as well as in regulation of disease. Monocytes, macrophage and dendritic cells serve main functions in the immune system; phagocytosis, antigen presentation, cytokine production and activation of the adaptive immune system. They play a central role in acute and chronic inflammation since they maintain the inflammatory condition by secretion of pro-inflammatory cytokines such as TNFα, IL-1β and IL-6. Anti-inflammatory drugs given systemically have adverse side effects, localize in healthy tissues or are rapidly excreted, a problem that can be circumvented by use of specific drug delivery systems. Therefore, targeted delivery to monocytes and dendritic cells is of great importance.

Cationic liposomes have been shown to exhibit a superior association with and retention to monocytes and dendritic cells either by cell membrane association, phagocytosis or endocytosis. These mechanisms may be induced by the complement system which is active in fresh human blood, but which is inactivated during storage or freezing of blood, plasma and serum. Several complement factors show biological half lives in the range of minutes.

Toll-like receptors (TLRs) are a class of receptors expressed on various cell types and play a key role in the innate immune system. Upon activation, TLRs activate signal transduction pathway involved in immune activation. Several mammalian TLRs and a number of their agonists have been identified. For example, guanine and uridine rich single-stranded RNA has been identified as a natural ligand for TLR7. In addition, several low molecular weight activators of TLR7 have been identified, including imidazoquinolines, and purine-like molecules. While TLR stimulation initiates a common signaling cascade (involving the adaptor protein MyD88, the transcription factor NFκB, and proinflammatory and effector cytokines), different TLRs are expressed by different cell types, however, TLR7 is mainly expressed in monocytes, plasmacytoid dendritic cells, myeloid dendritic cells and B-cells and are localized to the endosome membrane WO 2013/135800 discloses cationic liposomes for targeted delivery of drugs to monocytes in whole blood.

WO 2015/036044 discloses cationic liposomes for targeted delivery of TLR7 agonists to monocytes in whole blood.

Johansen et al. 2015 (Expert Opin. Drug Deliv. 2015; 12(7)) investigate the effect of liposome surface charge on monocyte targeting capability and conclude that cationic liposomes having zeta potentials in the 31-38 mV range exhibit superior monocyte targeting.

If a liposomal drug delivery system is to be used for immune modulation the formulation should preferentially be taken up by monocytes and dendritic cells, and not by other cell populations in the blood. There exists a need in the art to improve the specificity of delivery to monocytes and dendritic cells to further improve efficacy of immune-modulatory treatment and reduce side-effect of same.

### SUMMARY

Cationic liposomes of the present disclosure were prepared with the attempt to design liposomes with an ability to be recognized and selectively taken up by antigen presenting cells (APCs) like monocytes and dendritic cells but not by other cells in the blood. This has been accomplished by investigating the interactions between various liposome formulations and the specific cell populations in the blood.

A cationic liposome comprising at least one active ingredient, at least one cationic lipid, cholesterol and PEG conjugated to phospholipids, and which has a zeta potential in the range of 13-25 mV is provided herein. The inventors have surprisingly found that the cationic liposomes of the present disclosure exhibit a significant improvement in monocyte targeting. Furthermore, dendritic cells are targeted by the cationic liposomes of the present disclosure. The cationic liposome uptake is improved in respect to (i) fraction of monocytes taking up cationic liposomes, (ii) amount of cationic liposome taken up by each monocytes and (iii) selectivity towards monocytes over lymphocytes and B cells. Furthermore, the liposomes of the present disclosure aggregate less in human plasma as compared to non-PEG cationic liposomes.

In addition, the cationic liposomes of the present disclosure provide enhanced cytokine induction in human whole blood when loaded with a TLR7 agonist having a structure according to Formula IA. Furthermore, the cationic liposomes of the present disclosure comprising the TLR7 agonist according to Formula (IA) demonstrate a better toxicity score and immunogenicity score as compared to the non-PEG cationic liposomes. The inventors have demonstrated that co-administration of the cationic liposomes of the present disclosure with an anti-cancer agent result in improved efficacy in cancer treatment of colon cancer in a mouse model.

According to a first aspect, the present disclosure concerns a cationic liposome comprising cholesterol in the range of 0-40 mol %, PEG conjugated to a phospholipid in the range of 1-10 mol %, at least one cationic lipid and at least one active ingredient, wherein the remaining components are phospholipids and wherein the zeta potential is in the range of 13-25 mV.

The active ingredient may be an immunomodulatory agent, which may be an immunostimulating compound or an immunosuppressive compound. In a particular embodiment of the present disclosure, the active ingredient is a TLR7 agonist having a structure according to Formula IA.

According to a second aspect, the present disclosure concerns a lipid-based delivery system for targeting monocytes and dendritic cells in fresh whole blood, said system providing delivery to and release of at least one active ingredient to the targeted monocyte, and said system comprising cholesterol in the range of 0-40 mol %, PEG conjugated to a phospholipid in the range of 1-10 mol %, at least one cationic lipid and at least one active ingredient, wherein the remaining components are phospholipids and wherein the zeta potential is in the range of 13-25 mV, said system allowing the formation of cationic liposomes, at which said cationic liposomes preferentially adheres to monocytes and dendritic cells in fresh whole blood when compared to adherence to granulocytes, T-lymphocytes, B-lymphocytes and/or NK cells in fresh whole blood.

According to a third aspect, the present disclosure concerns a pharmaceutical composition comprising the cationic liposomes for use in prophylaxis, treatment or amelioration of cancer, an infectious disease, an inflammatory condition or disease, an autoimmune disease or allergy.

According to a fourth aspect, the present disclosure provides a method for in vitro activation or inhibition of monocytes and dendritic cells is provided, comprising the steps of: (i) providing fresh blood, (ii) administering the cationic liposome to said fresh blood, and (iii) allowing said cationic liposome to react.

According to a fifth aspect, the present disclosure provides a method for in vivo activation or inhibition of monocytes and dendritic cells in a subject is provided, comprising administering the cationic liposome to said subject in an amount sufficient to activate or inhibit said monocytes and dendritic cells.

According to a sixth aspect, the present disclosure provides a method for ex vivo activation or inhibition of monocytes and dendritic cells is provided, comprising the steps: (i) providing fresh blood from a subject, (ii) administering the cationic liposome to said fresh blood, (iii) allowing the cationic liposome to react with said fresh blood, and (iv) reintroducing said blood into the circulation of said subject.

Further advantages of the present disclosure include; (a) improved shelf life of the cationic liposome due to reduced liposome fusion with subsequent enlarged liposomes and (b) easier production of the cationic liposomes due to easier extrusion.

### DESCRIPTION OF DRAWINGS

FIG. 1:

Structure of the formulated TLR7 agonists 1v270 (also termed TMX-201) used for liposome preparations (Formula (IA), M=1085.4 g/mol, Name: 2-(4-((6-amino-2-(2-methoxyethoxy)-8-oxo-7H-purin-9(8H)-yl)methyl)benzamido) ethyl 2,3-bis(oleoyloxy)propyl phosphate).

FIG. 2:

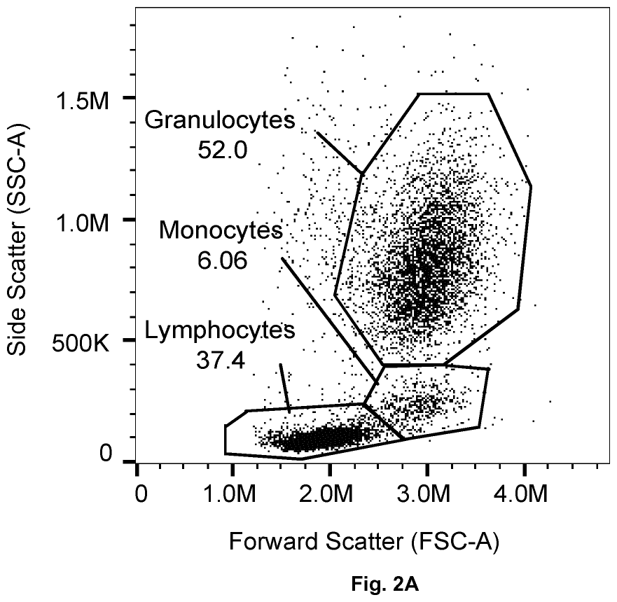

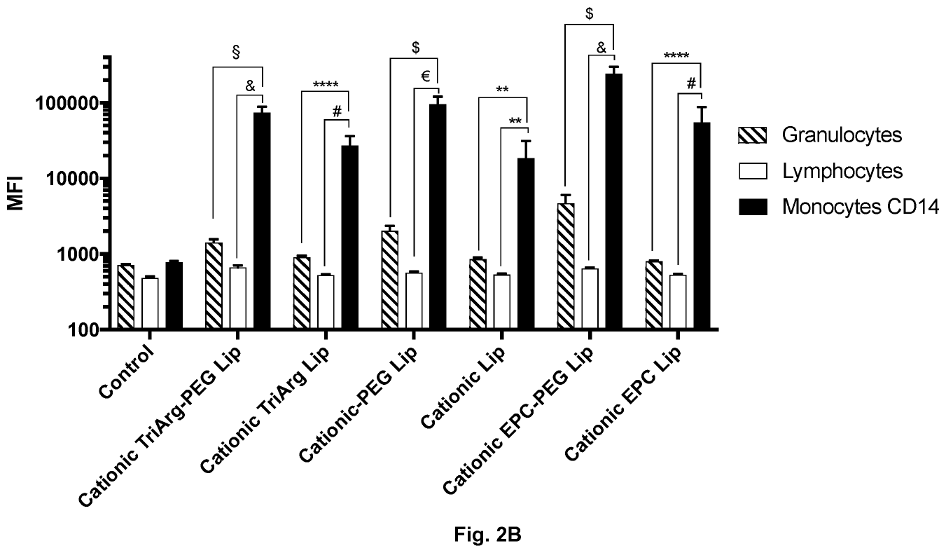

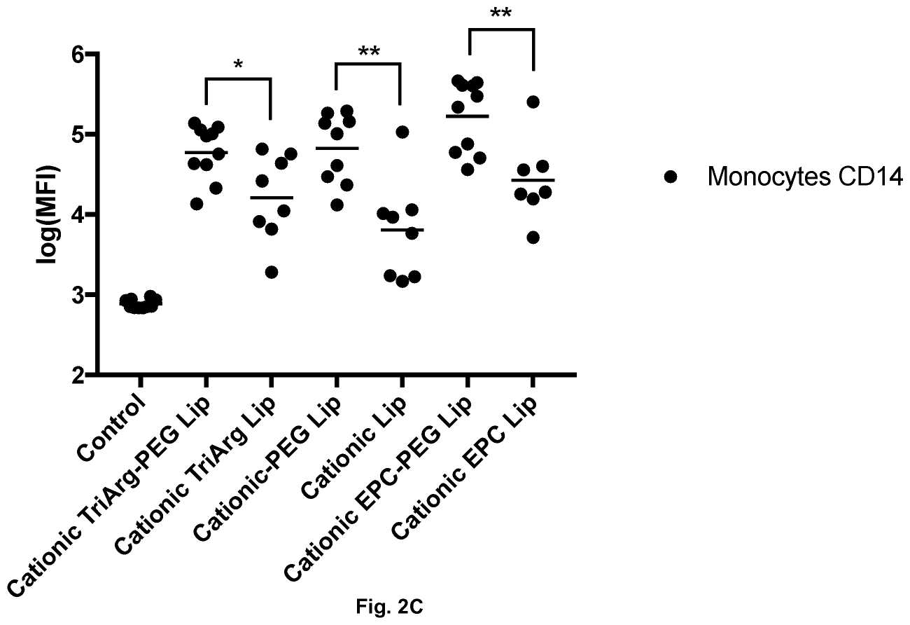

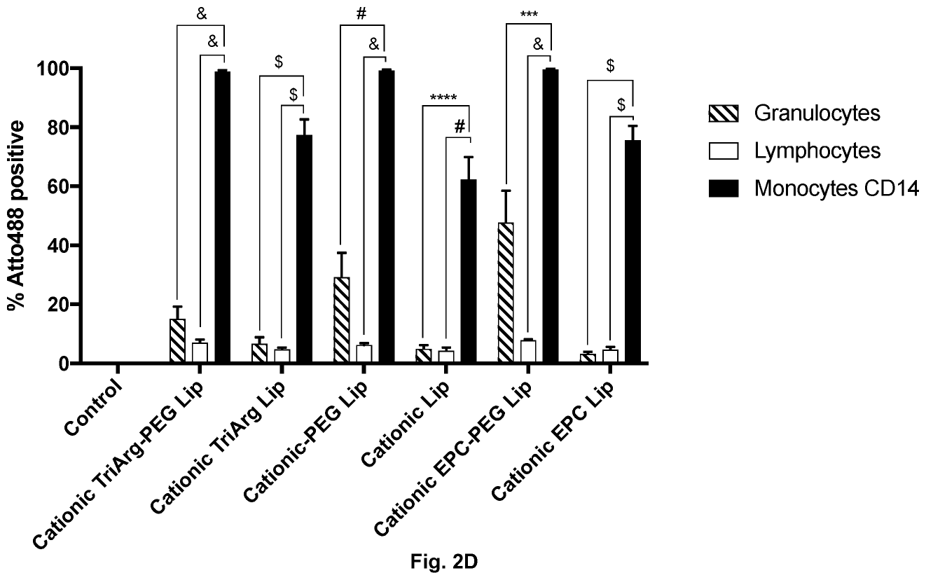

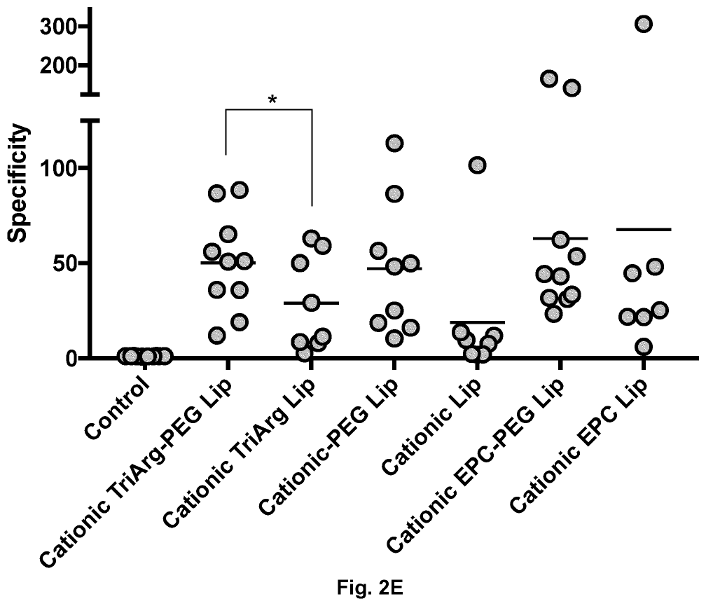

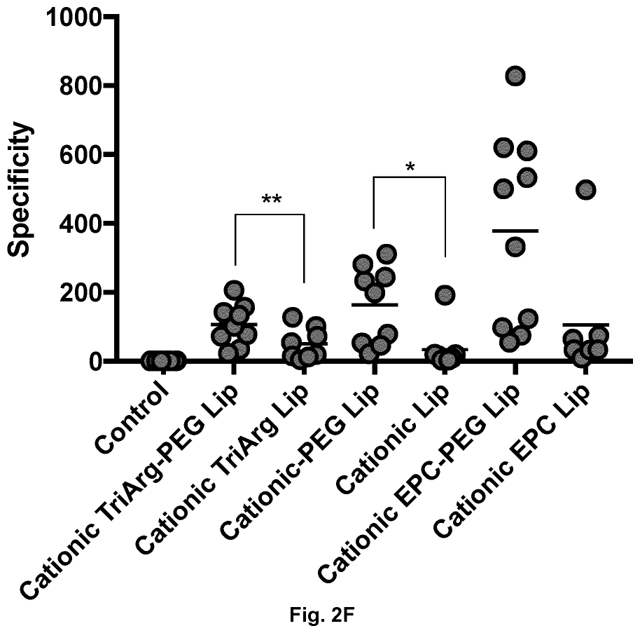

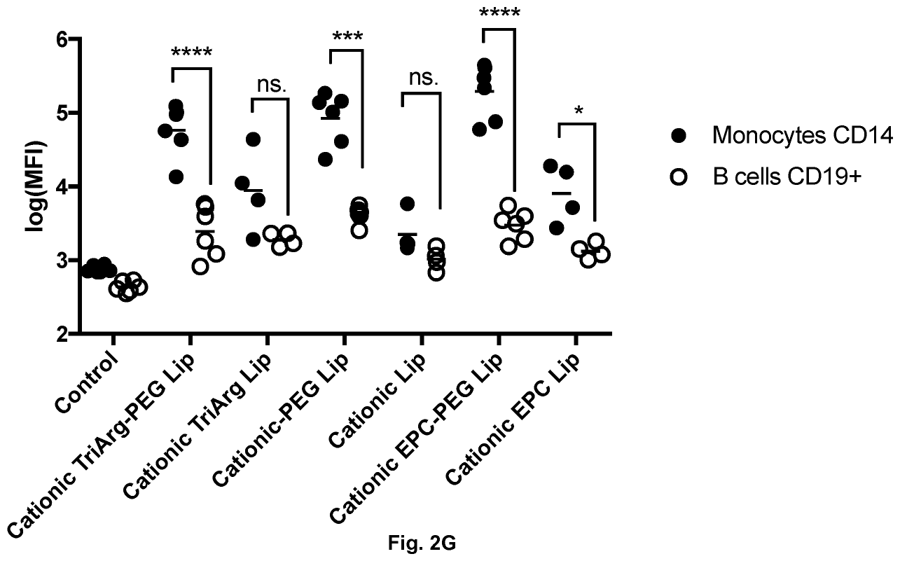

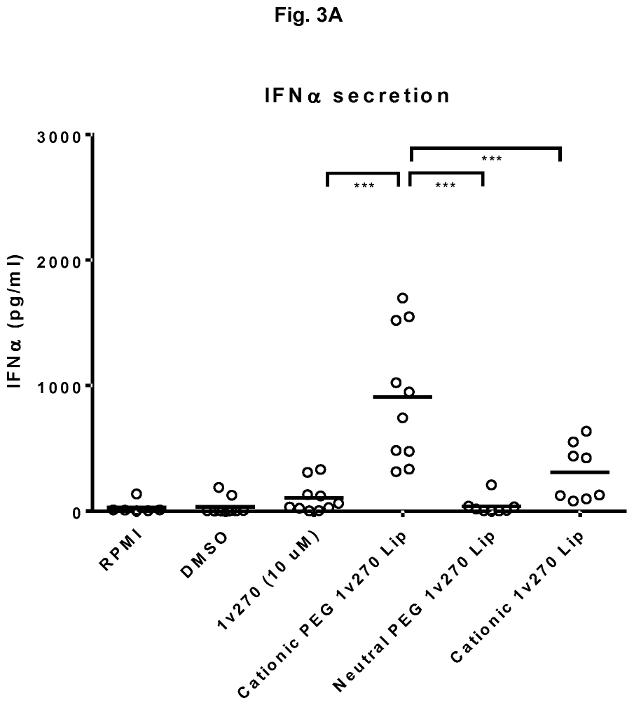

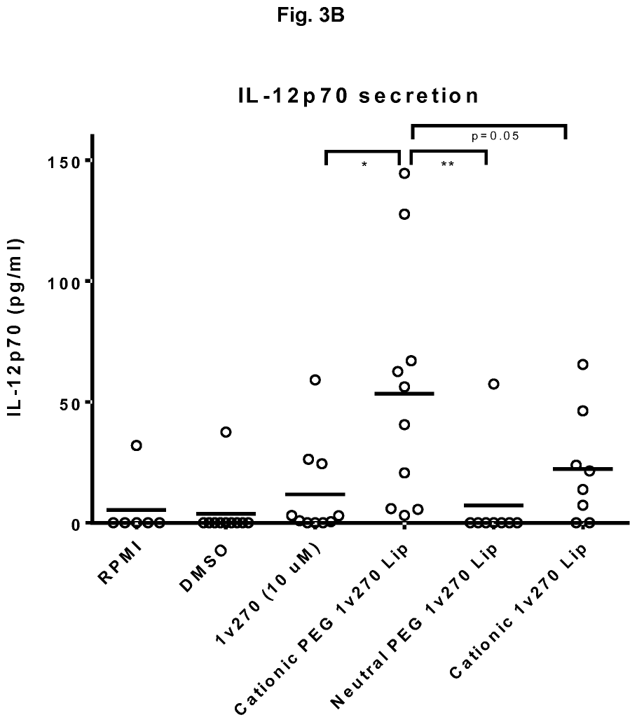

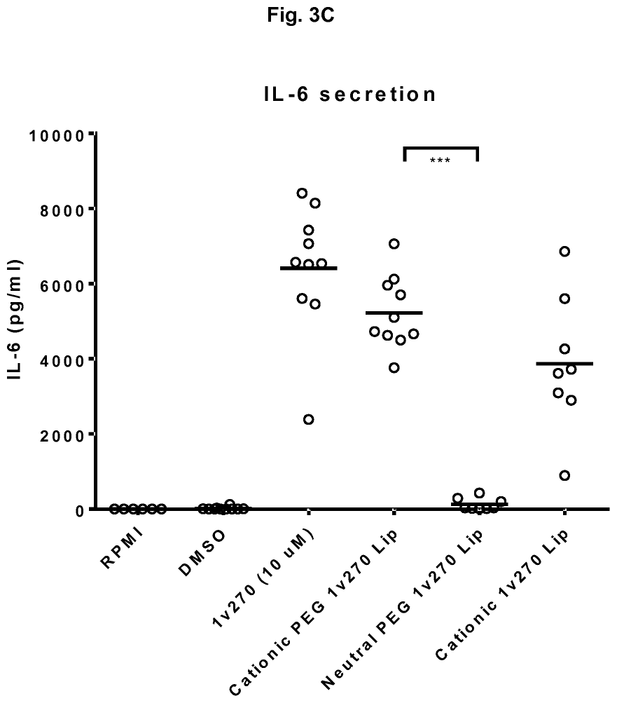

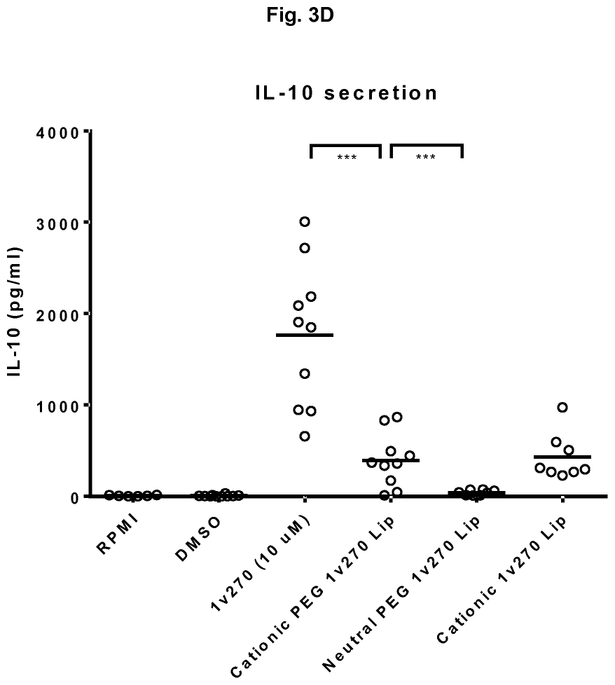

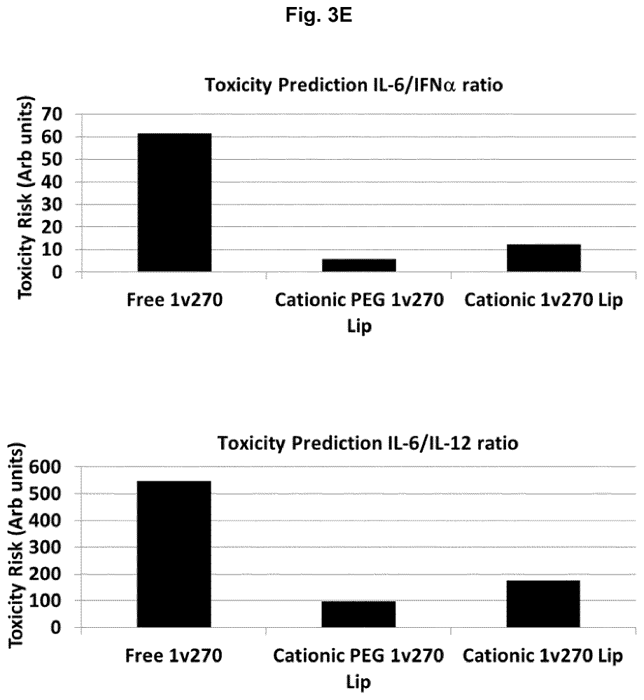

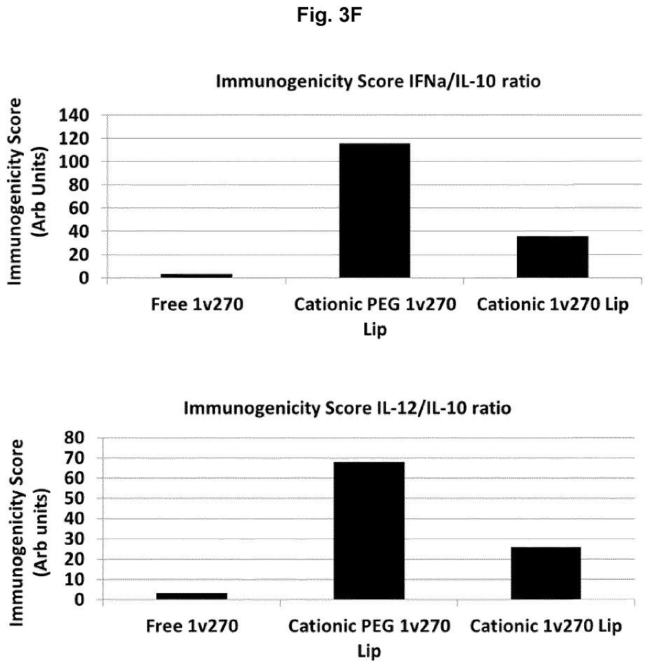

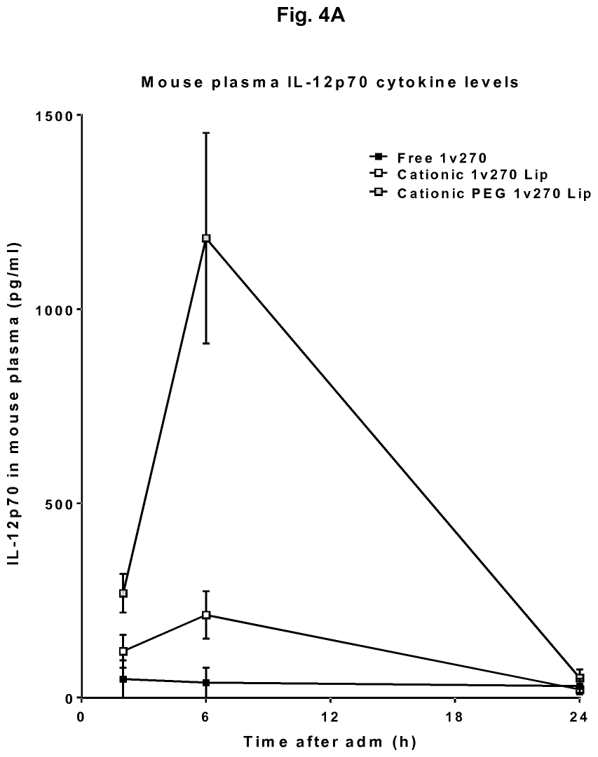

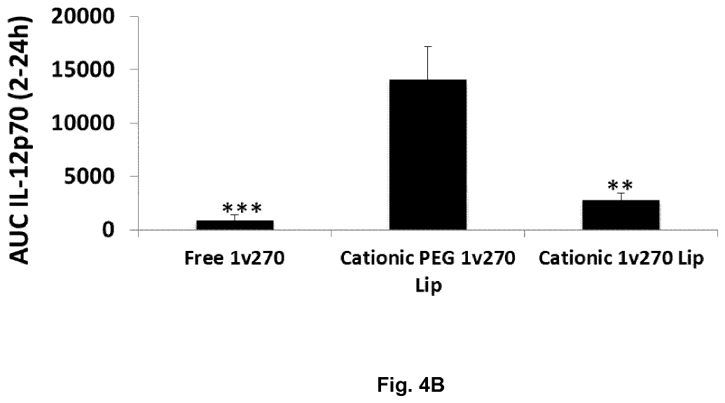

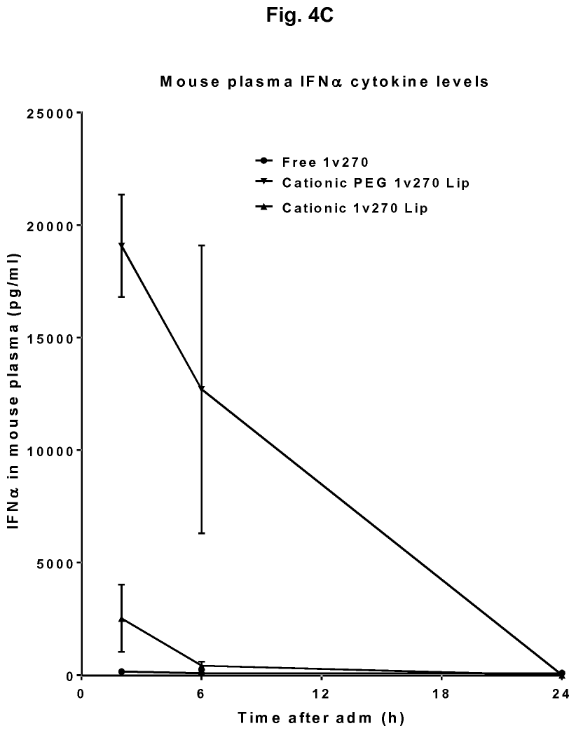

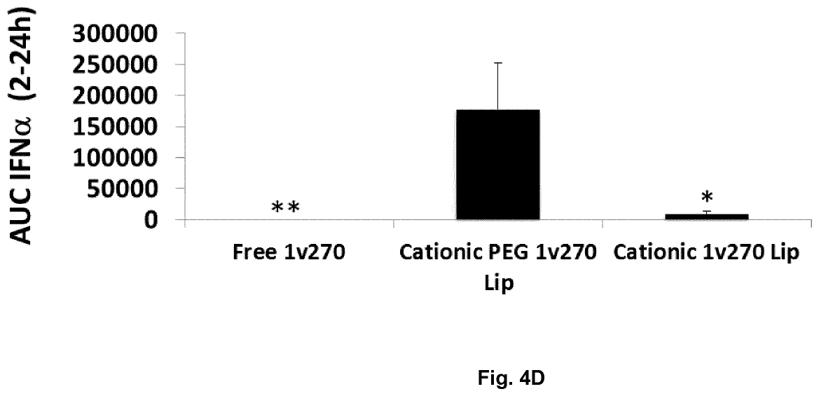

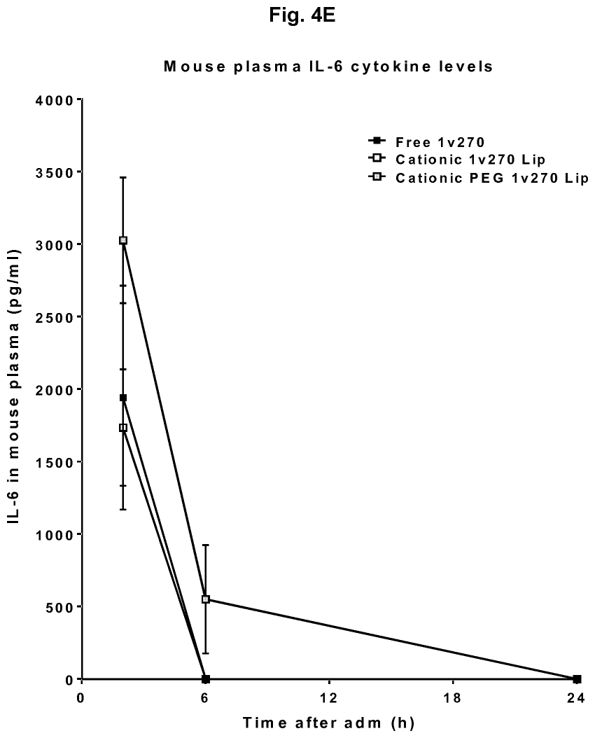

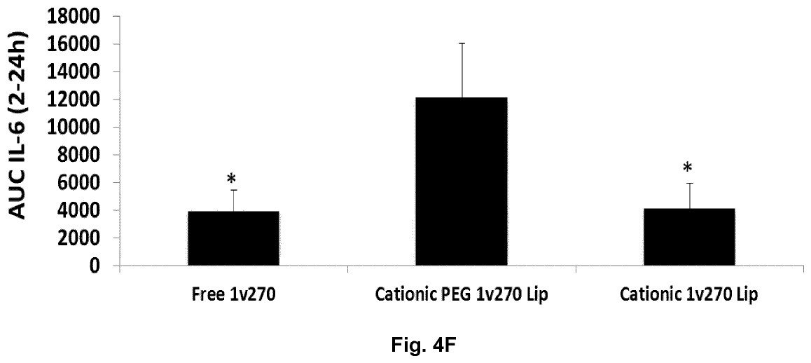

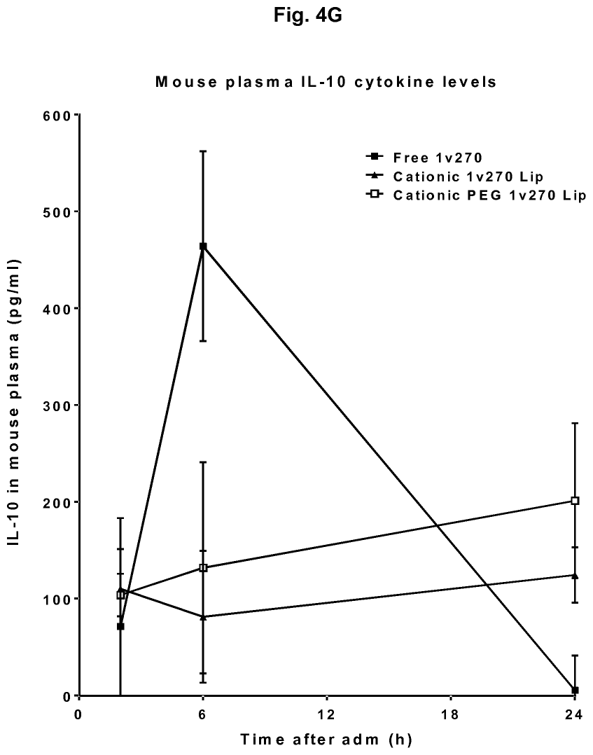

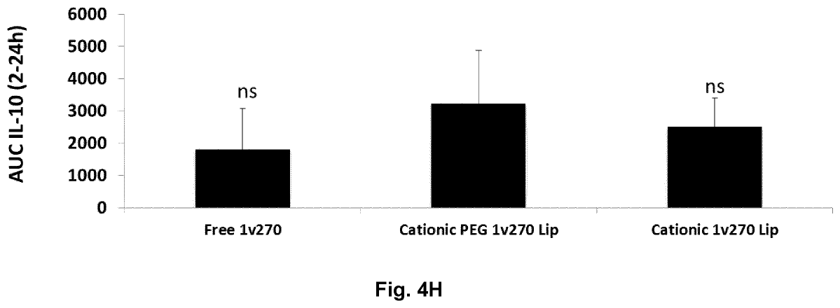

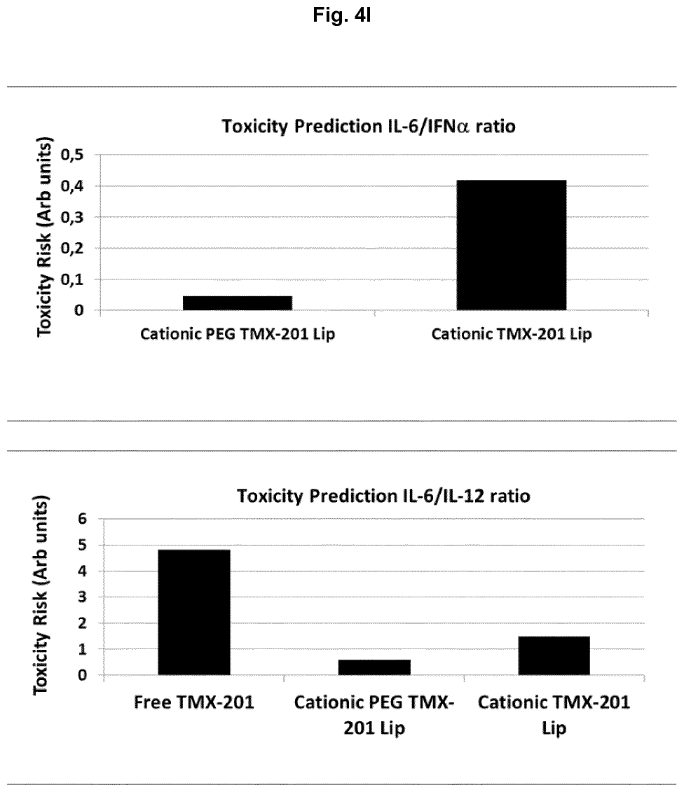

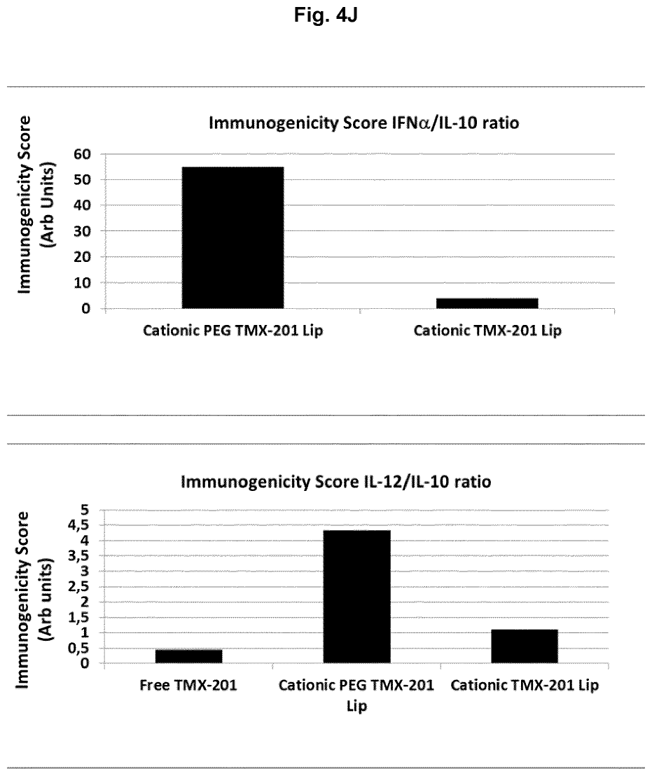

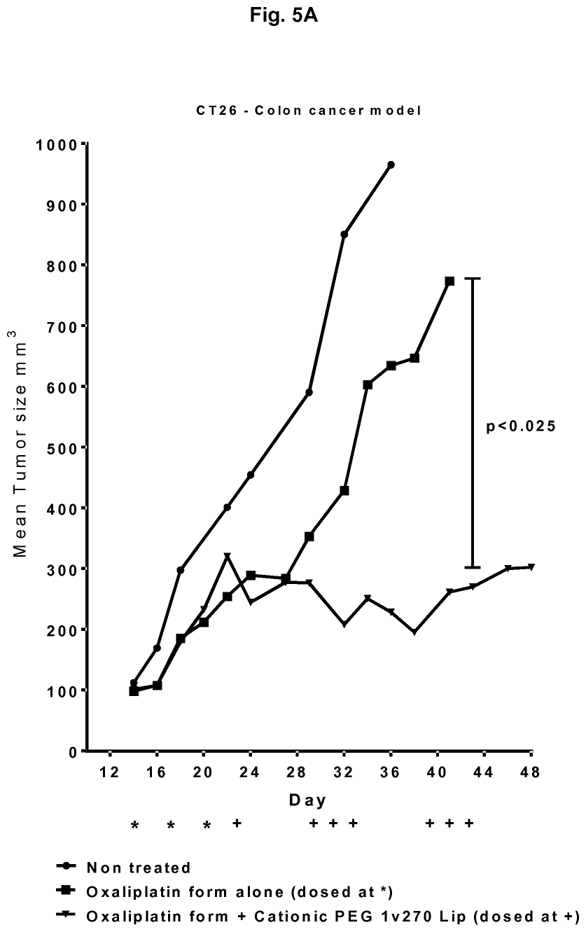

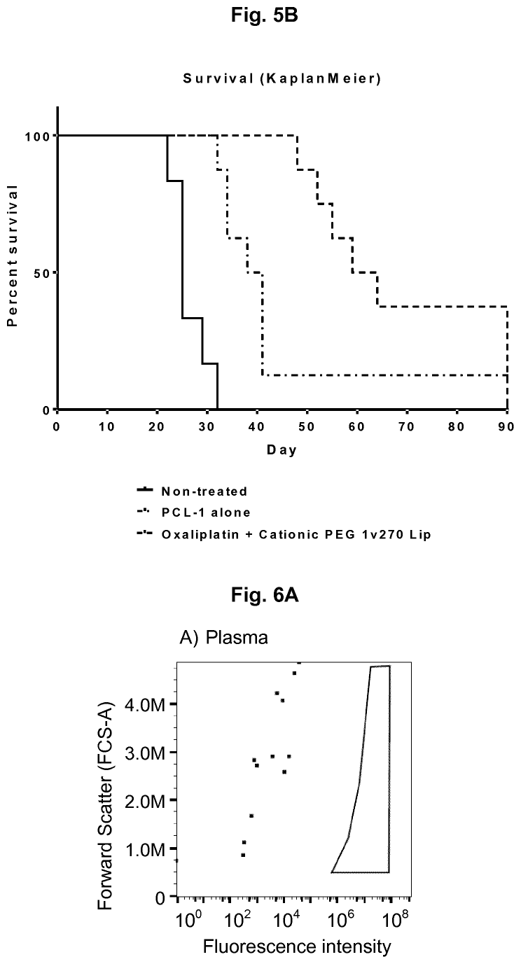

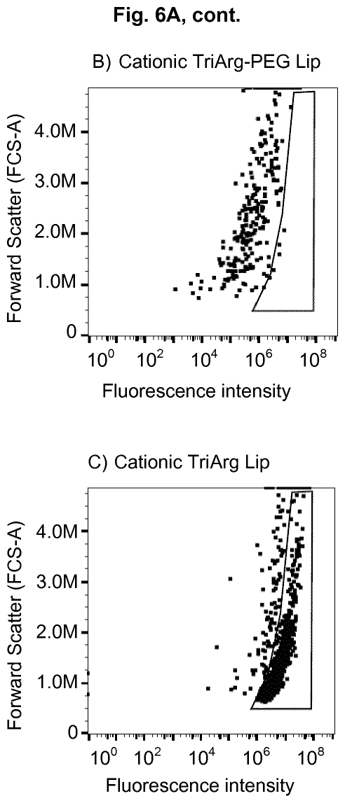

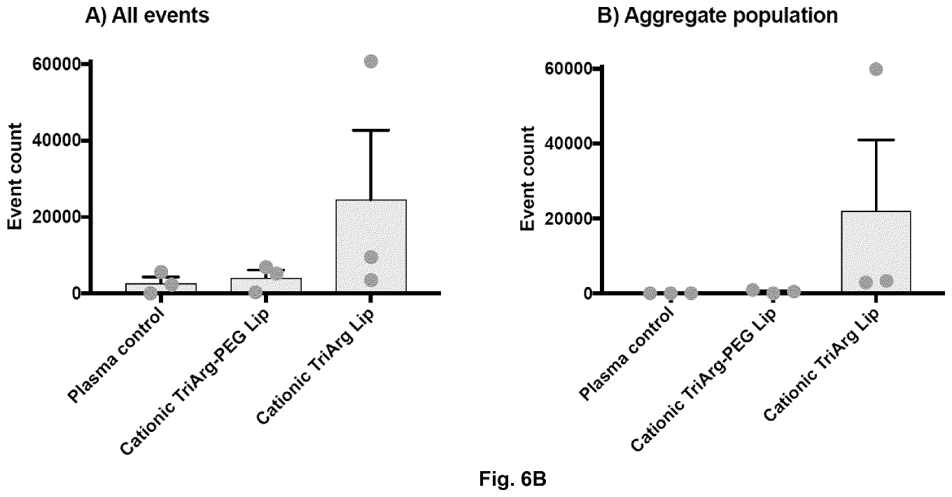

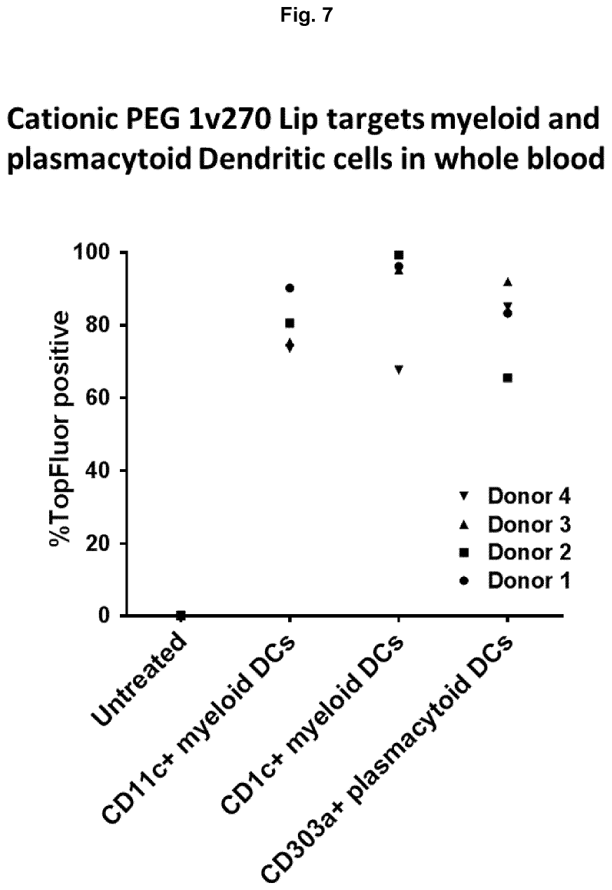

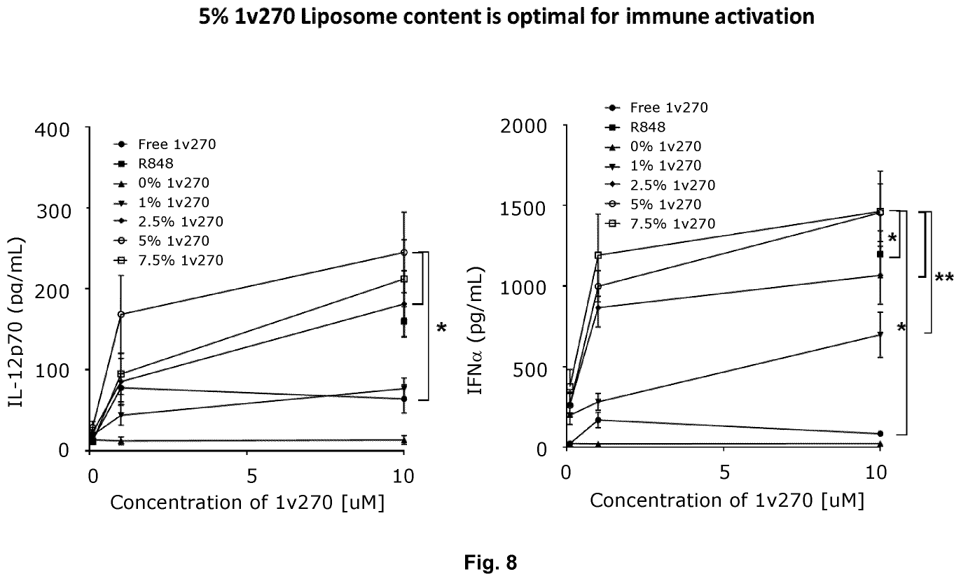

FIG. A-G compares uptake in leukocytes of the liposome formulations Cationic TriArg-PEG Lip, Cationic TriArg Lip, Cationic-PEG Lip, Cationic Lip, Cationic EPC-PEG Lip and Cationic EPC Lip (refer to Table 1 for liposome compositions) as described in example 4 (n=7-10). For whole FIG. 2, controls are fluorescent signal from cells treated with solvent PBS but no liposomes.

A) Gating strategy for examining monocytes, lymphocytes and granulocytes based on a forward scatter versus side scatter plot to distinguish leukocytes depending on morphological differences. CD14 positive cells were gated for within the monocyte gate and CD19 positive B cells within the lymphocyte gate.

B) Uptake of cationic liposomes in CD14+ monocytes, granulocytes and lymphocytes. All liposomes showed strong monocyte uptake compared to lymphocytes and granulocytes, with the Median Fluorescence Intensity (MFI) being 50-100-fold higher for monocytes than lymphocytes and granulocytes. Statistical test is a multiple t-test on log transformed data corrected with the Holm-Sidak method. Statistical significance is denoted with adjusted p-values: **p<0.005, ****p<5×10⁻⁵, #p<5×10⁻⁶, $p<5×10⁻⁷, €p<5×10⁻⁹, § p<5×10⁻¹⁰, &p<5×10⁻¹².

C) Comparison of monocyte targeting for PEGylated liposomes compared to non-PEGylated liposomes show a significantly higher uptake in monocytes for the PEGylated liposomes compared to non-PEGylated liposomes independent of the cationic component (test type is paired t test, *p<0.05, **p<0.005).

D) For the same set of liposomes as in figure B and C, the number of cells positive for liposome uptake as determined by Atto488 fluorescence over a control sample without liposomes is analysed and plotted as percentage of positive cells within each cell population. PEGylated liposomes all show nearly 100% of monocytes being positive to liposomes whereas non-PEGylated liposomes show uptake in 60-80% of monocytes. Association with granulocytes is maximum 50%, while no liposome formulation gives above 10% association with lymphocytes. Statistics are multiple t-test adjuset for multiple comparisons with the Holm-Sidak method. Adjusted p values are denoted with same symbols as in figure C.

E) Specificity of liposomes towards monocytes over granulocytes, calculated as MFI_{monocytes}/MFI_{granulocytes}. Test type is paired t test, *p<0.05.

F) Specificity of liposomes towards monocytes over lymphocytes, calculated as MFI_{monocytes}/MFI_{lymphocytes}. Test type is paired t test, *p<0.05, **p<0.005.
G) Uptake for monocytes compared to B-cells (CD19+) show a strong preference for monocyte targeting for PEGylated liposomes whereas non-PEGylated liposomes show no significant or weakly significant monocyte preference over B-cells (n=4-6, Paired T-test, *p<0.05, ***p<0.0005, ****p<5×10⁻⁵).

FIG. 3:

Induction of cytokines from whole blood after treatment and incubation for 24 h. For all figures statistical analyses was made using students T-test, *p<0.05, **p<0.01, ***p<0.005, n=8-10. Test samples were the free TLR7 agonist 1v270 (1v270), the PEGylated cationic formulation with the immune stimulating 1v270 TLR7 agonist (Cationic PEG 1v270 Lip), a non-targeting neutral PEGylated liposome with 1v270 TLR7 agonist (Neutral PEG 1v270 Lip) and a non-PEGylated cationic formulation with 1v270 (Cationic 1v270 Lip). Composition and properties can be seen in Table 1. RPMI is control sample only with addition of media and DMSO is a vehicle control as solvent for 1v270.

A) Secretion of the anti-viral and anti-cancer cytokine IFNα from treated cells showed significantly higher levels from the Cationic PEG 1v270 Lip formulation than any other treatment.

B) Secretion of the anti-cancer and Th1-inducing cytokine IL-12p70 from treated cells showed significantly higher levels from the Cationic PEG 1v270 Lip formulation than any other treatment.

C) Secretion of the pro-inflammatory cytokine IL-6 from treated cells showed a tendency to the free 1v270 compound as most potent, with slightly lower levels from Cationic liposomes and very low levels from Neutral liposomes.

D) Secretion of the immunosuppressive cytokine IL-10 from treated cells showed significantly higher levels from the 1v270 compound alone than all liposomes.

E) Based on cytokine secretions the toxicity prediction was determined by comparing the ratio of toxicity related IL-6 and levels of beneficial anti-tumor cytokines IFNα and IL-12p70.

F) Based on cytokine secretions the immunogenicity score was determined by comparing the ratio of beneficial anti-tumor cytokines IFNα and IL-12p70 and the immunosuppressive and in relation to cancer immunotherapy less beneficial cytokine IL-10.

FIG. 4:

Balb c mice were injected intravenously with 200 nmol/mouse of the free 1v270 compound, the Cationic PEG 1v270 Lip formulation and the Cationic 1v270 Lip formulation. Blood samples were drawn at 2, 6 and 24 h, and IL-12p70 measured by ELISA and concentration shown in FIG. 5A. Area Under the Curve (AUC) was calculated for each treatment and shown in FIG. 5B. C) Show analyses of IFNα measured in the same plasma samples, and shows a very strong induction of IFNα from the cationic PEG 1v270 Lip formulation compared to both free 1v270 and the Cationic 1v270 Lip formulation. AUC was calculated for each treatment and shown in FIG. 5D. E) Shows measurement of IL-6 at the same timepoints including F) calculation of the AUC for quantitative comparison. G) showing measurement of IL-10 from the mouse plasma samples with H) calculated AUC. The AUC values calculated for each cytokine were used to determine I) the Toxicity Prediction with the IL-6/IFNα and IL-6/IL-12p70 ratios, and J) the immunogenicity score by the IFNα/IL-10 and IL-12p70/IL-10 ratios shown in arbitrary units. (n=5 mice/group, Students T-test was used, *p<0.05, **p<0.01, *** p<0.005, ns=non significant, error bars show SE).

FIG. 5:

Mouse tumor study in the CT26 syngenic colon cancer model. CT26 cells were placed sc on Balb c mice on day 1, and treatment started at day 14 with 8 mg/kg Oxaliplatin formulation at an approximate tumor size of 100 mm³. This formulation was given two times more at the same dose on day 17 and 20. Cationic PEG 1v270 Lip immunotherapy was given with 40 nmol/20 g mouse of the 1v270 TLR7 agonist on days 23, 29, 31, 33, 43, 45 and 47. Mice with weight other than 20 g was adjusted to receive 2 nmol/g. Tumors were measured 2-3 times a week.

A) The mean tumor size is shown as the mean for each treatment group of 8 mice. (n=8 mice/group, Wilcoxon rank sum test showed a statistical better tumor inhibition for the combined oxaliplatin formulation with Cationic PEG 1v270 Lip immunotherapy with p<0.025).

B) The same study expressed in a KaplanMeier survival curve, showing the time and sacrifice of mice due to tumor burden. One mouse in the oxaliplatin formulation alone group experienced complete remission, whereas three of 8 mice experienced complete remission in the combined treatment group. Non-treated mice received control PBS administered iv.

FIG. 6:

A) Aggregation of liposomes in plasma with and without PEG. Aggregation of liposomes after incubation in human plasma was investigated by flow cytometry. Horizontal axis show fluorescence of TopFI, vertical axis show forward scatter—both of which are related to the particle size, but only particles originating from liposomes will be fluorescent. Pure plasma containing very few events of various sizes but no fluorescence (Panel A). Cationic PEGylated liposomes are added to the sample, giving a small increase in the number of events collected (Panel B). Cationic non-PEGylated liposomes are added and show a very large increase in the number of events at high forward scatter and a distinct population with high fluorescence intensity demonstrating the liposomes have created large aggregates (Panel C).

B) Quantification of aggregation dependent on presence of PEG on liposomes based on Flow cytometry studies from 3 donors showed a strong aggregation for non-PEG liposomes using total counts of events (Panel A), or after gating of the specific aggregated population (Panel B). Plasma control is pure human plasma prepared by centrifugation of the anti-coagulated blood (n=3, error bars show SEM).

FIG. 7:

Targeting to dendritic cells with cationic liposomes in whole human blood from 4 healthy donors. Cationic PEG 1v270 Liposomes were added to fresh blood and incubated for 60 min, and subsequently analyzed by flow cytometry using the gating strategy described in example 9. Between 60%-100% of dendritic cells were positive for liposome uptake shown by the percentage of DCs positive for TopFluor. The two myeloid DC subsets were analyzed (CD11c myeloid DCs, CD1c myeloid DCs and CD303a plasmacytoid DCs), untreated control is prepared as blood sample without addition of liposomes. (n=4).

FIG. 8:

Optimization of 1v270 content in cationic liposomes. Cationic PEG liposomes containing 1v270 in varying amounts from 0-7.5% were prepared according to Table 2 and tested with incubation in whole blood with rotation for 60 min followed by 24 h incubation to measure cytokine secretion. The same amount of 1v270 was added for each formulation independent of 1v270 (1-7.5%) content for each liposome, except when there was no 1v270 present. E.g. for the formulation with 1% 1v270, five times more liposome was added as with the 5% 1v270 liposome to reach equal amounts of 1v270 added. R848 was included as a control at 10 μM. 5% 1v270 was the optimal content in liposomes shown by the ability of this formulation to induce the most potent IL-12p70 (left) and IFNα (right) cytokine response. (n=5 donors, *p<0.05, **p<0.01).

### DEFINITIONS

A “liposome” in the present application and claims denotes an artificial prepared vesicle made of at least one lipid bilayer.

The term “preferentially adheres” as used in the present context means that the cationic liposomes according to the present disclosure adheres to monocytes and dendritic cells in fresh whole blood to an extent which is at least 1.5 times larger than the adherence to granulocytes and 3 times larger than lymphocytes in fresh whole blood, for example at least 2 times larger than the adherence to granulocytes and 5 times larger than lymphocytes, for example at least 4 times larger than the adherence to granulocytes and 8 times larger than lymphocytes, for example at least 5 times larger than the adherence to granulocytes and 10 times larger than lymphocytes, preferably at least 10 times larger than the adherence to granulocytes and 25 times larger than lymphocytes, more preferably at least 20 times larger than the adherence to granulocytes and 40 times larger than lymphocytes, in fresh whole blood.

The term “fresh whole blood” in the present context means that the blood in question has been drawn from a mammal within no more than 60 minutes, such as no more than 30 minutes, preferably no more than 15 minutes.

The term “zeta potential” in the present context describes the electric potential at the location of the slipping plane of a colloidal particle in solution versus a point in the bulk fluid away from the interface. Liposomes are generally considered cationic when the zeta potential is above 10. The zeta potential as defined in the present disclosure is measured in 300 mM glucose, 10 mM HEPES, 1 mM CaCl₂ in MilliQ water, pH 7.4, according to the conditions as disclosed in Example 3. The same zeta potential is obtained when measuring in 10% Sucrose, 10 mM HEPES, 1 mM CaCl₂ in MilliQ water, pH 7.4.

The term “immunomodulatory agent”, as used herein, refers to an agent which is capable of modulating an immunological response.

The term “immunostimulating compound” as used in the present context means capable of stimulating the innate and/or adaptive immune system.

The term “immunosuppressive compound” as used in the present context means capable of down-modulating an immunological response.

The term “mol %”, as used herein, is defined as the molar amount of a constituent, divided by the total molar amount of all constituents in a mixture, multiplied by 100.

The term “PEG”, as used herein, refers to the polyether compound polyethylene glycol. PEG is currently available in several sizes and may e.g. be selected from PEG350, PEG550, PEG750, PEG1000, PEG2000, PEG3000, PEG5000, PEG10000, PEG20000 and PEG30000. The number refers to the molecular weight of the ethylene units.

The term “physiological conditions”, as used herein, refers to conditions simulating in vivo conditions or being in vivo conditions. Physiological systems are generally considered to be comprised of an aqueous system having a pH of about 7.

The term “monovalent”, as used herein, refers to a chemical compound, herein a cationic lipid, having a charge of +1.

The term “divalent”, as used herein, refers to a chemical compound, herein a cationic lipid, having a charge of +2.

The term “trivalent”, as used herein, refers to a chemical compound, herein a cationic lipid, having a charge of +3.

The term “multivalent”, as used herein, refers to a chemical compound, herein a cationic lipid, having a charge of >+3.

The term “prophylaxis”, as used herein, refers to prevention of a disease or prevention of spreading of a disease.

The term “treatment”, as used herein, refers to the combating of a disease or disorder. “Treatment” or “treating,” as used herein, includes any desirable effect on the symptoms or pathology of a disease or condition as described herein, and may include even minimal changes or improvements in one or more measurable markers of the disease or condition being treated. “Treatment” or “treating” does not necessarily indicate complete eradication or cure of the disease or condition, or associated symptoms thereof.

The term “amelioration”, as used herein, refers to moderation in the severity of the symptoms of a disease or condition. Improvement in a patient's condition, or the activity of making an effort to correct, or at least make more acceptable, conditions that are difficult to endure related to patient's conditions is considered “ameliorative” treatment.

### DETAILED DESCRIPTION

It will be clear for the person skilled in the art, that aspects and/or embodiments as described herein may be combined.

The present disclosure relates to a cationic liposome comprising cholesterol in the range of 0-40 mol %, PEG conjugated to a phospholipid in the range of 1-10 mol %, at least one cationic lipid and at least one active ingredient, wherein the remaining components are phospholipids. The zeta potential of the liposomes is usually in the range of 13-25 mV.

In one embodiment, the cationic liposome comprises:
a. between 25-35 mol % cholesterol,
b. between 3-7 mol % PEG conjugated to a phospholipid,
c. at least one cationic lipid, and
d. at least one active ingredient,

wherein the remaining components are phospholipids and wherein the zeta potential is in the range of 13-25 mV.

In a particular embodiment, the cationic liposome comprises:
a. between about 30-40 mol % POPC, such as about 35 mol % POPC,
b. between about 25-35 mol % cholesterol, such as about 30 mol % cholesterol,
c. between about 3-7 mol % DOPE-PEG2000, such as about 5 mol % DOPE-PEG2000,
d. between about 20-30 mol % DOTAP, such as about 25 mol % DOTAP, and
e. between about 2.5-7.5 mol % of the TLR7 agonist 1v270, such as about 5 mol % 1v270,

wherein the zeta potential is in the range of 13-25 mV.

In another embodiment, the cationic liposome comprises:
a. between about 50-60 mol % POPC, such as about 54 mol % POPC,
b. between about 25-35 mol % cholesterol, such as about 30 mol % cholesterol,
c. between about 3-7 mol % DOPE-PEG2000, such as about 5 mol % DOPE-PEG2000,
d. between about 4-8 mol % TriArg, such as about 6 mol % TriArg, and
e. between about 2.5-7.5 mol % of the active ingredient, such as a TLR7 agonist,

wherein the zeta potential is in the range of 13-25 mV.

In one embodiment, the cationic liposome comprises:
a. between about 35-45 mol % POPC, such as about 40 mol % POPC
b. between about 25-35 mol % cholesterol, such as about 30 mol % cholesterol,
c. between about 3-7 mol % DOPE-PEG2000, such as about 5 mol % DOPE-PEG2000,
d. between about 15-25 mol % EPC, such as about 20 mol % EPC, and
e. between about 2.5-7.5 mol % of the active ingredient, such as a TLR7 agonist,

wherein the zeta potential is in the range of 13-25 mV.

### Cationic Liposome Structure and Characteristics

The cationic liposomes disclosed herein preferentially adhere to monocytes and dendritic cells over lymphocytes.

For monocyte and dendritic cell specific targeting of drugs, cationic liposomes will likely be phagocytosed by the monocyte or dendritic cell, and the associated drug will be released inside the monocyte or dendritic cell, allowing the drug to exert its intracellular function. For immunostimulating compounds like e.g. agonists towards intracellular receptors like pattern recognition receptors (PRRs), these molecules will be released once inside the cell, and activate the relevant receptor, which may result in immune stimulatory monocytes and dendritic cells.

### Active Ingredient

The cationic liposome of the present disclosure includes an active ingredient, typically a drug substance or composition. Normally, one of the compartments selected among the interior aqueous compartment, a hydrophobic bilayer, and a polar inter-phase of the inner and outer leaflet of the cationic liposome carry said at least one active ingredient. As an example, the active ingredient according to Formula IA is localized in the hydrophobic bilayer of the cationic liposome.

The skilled person will generally be knowledgeable about the choice of active ingredient and the correct dosage thereof.

In one embodiment, the cationic liposome comprises one or more active ingredients, such as two or more, such as three or more, such as four or more, such as five or more.

In a particular embodiment, the active ingredient is an immunomodulatory agent.

In preferred embodiments, the active ingredient is in the form of an immunostimulating compound, preferably with the ability to stimulate the innate immune system.

An embodiment of the present disclosure is thus a cationic liposome, wherein said at least one active ingredient is an immunostimulating compound which is a ligand for intracellular proteins and/or receptors.

An embodiment of the present disclosure is a cationic liposome, wherein said intracellular proteins and/or receptors are selected from the group consisting of STING, TLR3, TLR7, TLR8, TLR9, NOD1, NOD2, NOD5, NALP1, NALP2, NALP3, NALP12, NALP14, IPAF, NAIP, CIITA, RIG-I, MDA5, and LGP2, preferably selected from STING, TLR3, TLR7, TLR8, TLR9, and NOD2, more preferably TLR7.

In a preferred embodiment, the immunomodulatory agent is a TLR7 agonist selected from any one of Formula (I), Formula (II), Formula (III) and Formula (IV).

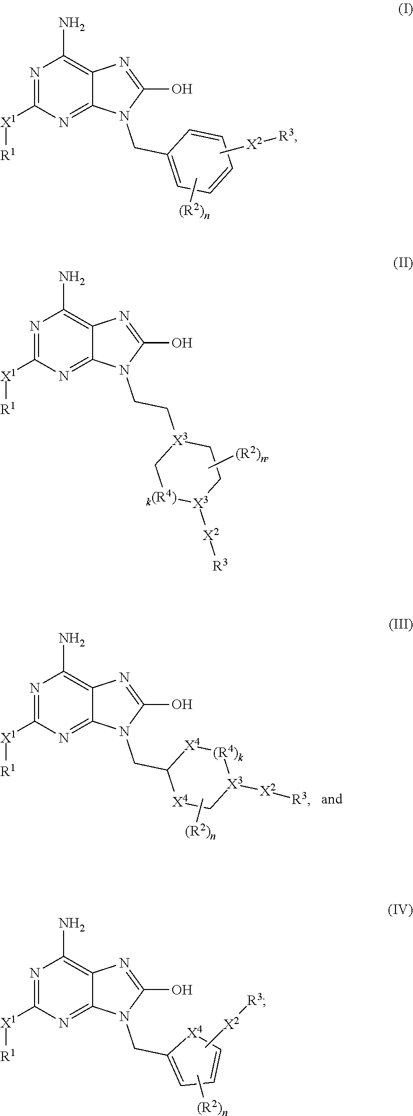

wherein X¹ is -0-, —S—, or —NR^{C};

R¹ is hydrogen, (C₁-C₁₀)alkyl, substituted (C₁-C₁₀)alkyl, C₆₋₁₀aryl, or substituted C₆₋₁₀aryl, C₅₋₉heterocyclic, substituted C₅₋₉heterocyclic;

R^{C} is hydrogen, C₁₋₁₀alkyl, or substituted C₁₋₁₀alkyl; or R^{C} and R¹ taken together with the nitrogen to which they are attached form a heterocyclic ring or a substituted heterocycli ring;

each R² is independently —OH, (C₁-C₆)alkyl, substituted (C₁-C₆)alkyl, (C₁-C₆)alkoxy, substituted (C₁-C₆)alkoxy, —C(0)-(C₁-C₆)alkyl (alkanoyl), substituted —C(0)-(C₁-C₆)alkyl, —C(0)-(C₆-C₁₀)aryl (aroyl), substituted —C(O)—(C₆-C₁₀)aryl, —C(0)OH (carboxyl), —C(0)0(C₁-C₆)alkyl (alkoxycarbonyl), substituted —C(0)0(C₁-C₆)alkyl, —NR^{a}R^{b}, —C(0)NR^{a}R^{b} (carbamoyl), halo, nitro, or cyano, or R² is absent;

each R^{a} and R^{b} is independently hydrogen, (C₁-C₆)alkyl, substituted (C₁-C₆)alkyl, (C₃-C₈)cycloalkyl, substituted (C₃-C₈)cycloalkyl, (C₁-C₆)alkoxy, substituted (C₁-C₆)alkoxy, (C₁-C₆)alkanoyl, substituted (C₁-C₆)alkanoyl, aryl, aryl(C₁-C₆)alkyl, Het, Het (C₁-C₆)alkyl, or (C₁-C₆)alkoxycarbonyl;

wherein the substituents on any alkyl, aryl or heterocyclic groups are hydroxy, C₁₋₆alkyl, hydroxyC₁₋₆alkylene, C₁₋₆alkoxy, C₃₋₆cycloalkyl, C₁₋₆alkoxy C₁₋₆alkylene, amino, cyano, halo, or aryl;

n is 0, 1, 2, 3 or 4;

X² is a bond or a linking group; and

R³ is a phospholipid comprising one or two carboxylic esters;

X³ is —N— or —CH—;

R⁴ is —CH2- or —CH(R<2>)-; and

k is 0 or 1;

X⁴ is —O—, —S—, —NH—, —N(R^{d})—, —CH₂—, or —CH(R²)—;

each R^{d} is independently —OH, (C₁-C₆)alkyl, substituted (C₁-C₆)alkyl, (C₁-C₆)alkoxy, substituted (C₁-C₆)alkoxy, —C(0)-(C₁-C₆)alkyl (alkanoyl), substituted —C(0)-(C₁-C₆)alkyl, —C(0)-(C₆-C₁₀)aryl (aroyl), substituted —C(O)—(C₆-C₁₀)aryl, —C(0)0(C₁-C₆)alkyl (alkoxycarbonyl), substituted —C(0)0(C₁-C₆)alkyl, —C(0)NR^{a}R^{b} (carbamoyl);

or a tautomer thereof;

or a pharmaceutically acceptable salt or solvate thereof.

It is to be understood that the ring system of formula (II) in some embodiments according to the present present disclosure is a piperidin ring with one heteroatom being an N atom and with the N-atom of the piperidin ring adjacent to X².

Also it is to be understood that the purine group in any of Formula (I), (II), (III), or (IV) is subject to tautomeric rearrangements.

In a particularly preferred embodiment, the immunomodulatory agent is a TLR7 agonist having a structure according to Formula (IA), wherein

X¹ is -0-;

R¹ is 2-methoxy-1-ethyl

R² is absent;

X² is carbonyl; and

R³ is 1,2-dioleoyl-sn-glycero-3-phosphoethanolamine (DOPE);

or a pharmaceutically acceptable salt or solvate thereof.

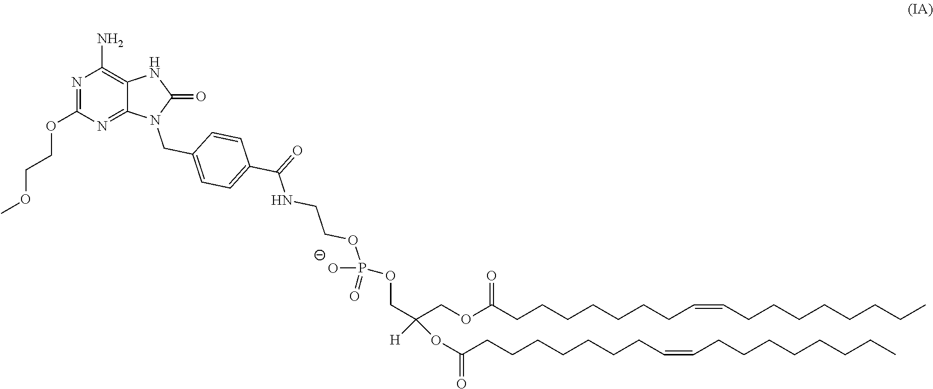 [SMILES: CCCCCCCC/C=C\CCCCCCCC(=O)OCC(COP(=O)([O-])OCCNC(=O)c1ccc(Cn2c(=O)[nH]c3c(N)nc(OCCOC)nc32)cc1)OC(=O)CCCCCCC/C=C\CCCCCCCC]

Compound according to Formula (IA) is also known in the literature as 1v270 and is known from e.g. U.S. Pat. No. 8,357,374.

In a preferred embodiment, the active agent is not a nanoprecipitated salt.

An embodiment of the present disclosure is a cationic liposome, wherein said at least one active ingredient is an immunosuppressive compound.

In one embodiment of the present disclosure, the active ingredient is not a nucleic acid, such as DNA, RNA or modified versions thereof.

In one embodiment, the cationic liposome comprises said active ingredient in the range of about 0.1-50 mol %, for example about 2-50 mol %, for example about 5-50 mol %, for example about 10-50 mol %, for example about 25-50 mol %, for example about 35-50 mol %, preferably the content of the active ingredient is about 5 mol %.

In one embodiment, the cationic liposome comprises said active ingredient in the range of about 0.1-50 mol %, for example about 0.1-40 mol %, for example about 0.1-30 mol %, for example about 0.1-20 mol %, for example about 0.1-10 mol %, such as about 0.1-7 mol %, preferably the content of the active ingredient is about 5 mol %.

In one embodiment, the cationic liposome comprises said active ingredient in the range of about 0.1-50 mol %, for example about 2-40 mol %, for example about 5-30 mol %, for example about 10-20 mol %, for example about 2.5-7.5 mol %, such as about 3-7 mol %, preferably the content of the active ingredient is about 5 mol %.

In one embodiment, the cationic liposome comprises said active ingredient in the range of about 0.1-15 mol %, for example about 1-15 mol %, for example about 2-15 mol %, for example about 4-15 mol %, for example about 6-15 mol %, for example about 8-15 mol %, for example about 10-15 mol %, for example about 12-15 mol %, preferably the content of the active ingredient is about 5 mol %.

In one embodiment, the cationic liposome comprises said active ingredient in the range of about 0.1-15 mol %, about for example 0.1-14 mol %, for example about 0.1-12 mol %, for example about 0.1-10 mol %, for example about 0.1-8 mol %, for example about 0.1-6 mol %, for example about 0.1-4 mol %, for example about 0.1-2 mol %, preferably the content of the active ingredient is about 5 mol %.

In one embodiment, the cationic liposome comprises said active ingredient in the range of about 0.1-15 mol %, for example about 0.5-12 mol %, for example about 1-10 mol %, for example about 2.5-7.5 mol %, such as about 3-7 mol %, preferably the content of the active ingredient is about 5 mol %.

In one embodiment, the cationic liposome comprises at least two immunomodulatory agents.

In one embodiment, the cationic liposome comprises at least one immunomodulatory agent and at least one antigen.

The antigen may e.g. be selected from the group consisting of a cancer antigen, a self- or autoimmune antigen, a microbial antigen, an allergen, or an environmental antigen.

### Zeta Potential Range

The zeta potential of the present present disclosure is measured at physiological conditions.

The cationic liposomes disclosed herein for specific monocyte and dendritic cell targeting show a zeta potential between 13-25 mV, preferably between 15-25 mV when measured on a ZetaPALS zeta potential analyzer (Brookhaven Instruments Coorporation, Holtsville, N.Y.) in a buffer consisting of 300 mM glucose, 10 mM HEPES, 1 mM CaCl₂ in MilliQ water, pH 7.4. The same zeta potential is obtained when measuring in 10% Sucrose, 10 mM HEPES, 1 mM CaCl₂ in MilliQ water, pH 7.4.

In one embodiment, the zeta potential is in the range of about 14-25 mV, such as 14-20 mV.

In a preferred embodiment, the zeta potential is in the range of about 15-25 mV, more preferably about 15-20 mV.

### Cationic Lipid

An embodiment of the present disclosure is a cationic liposome, wherein a part of the lipids is a cationic lipid.

Johansen et al. 2015 (Expert Expert Opin Drug Deliv. 2015; 12(7)) investigate the effect of liposome surface charge on monocyte targeting capability and report that incorporation of PEG in cationic liposomes result in reduced adherence to monocytes. The cationic liposomes of the present disclosure comprise a higher amount of cationic lipid and therefore have a higher zeta potential as compared to those of Johansen et al. and surprisingly show improved adherence to monocytes of the PEG containing cationic liposomes as compared to the non-PEG cationic liposomes.

The cationic lipids may e.g. be selected from monovalent cationic lipids, divalent cationic lipids, trivalent cationic lipids or multivalent cationic lipids.

Examples of monovalent cationic lipids include stearylamine (SA), lauryltrimethylammonium bromide, cetyltrimethylammonium bromide, myristyltrimethylammonium bromide, dimethyldioctadecylammonium bromide (DDAB), 30-[N-(N′,N′-dimethylaminoethane)-carbamoyl]cholesterol (DC-Cholesterol), 1,2-ditetradecanoyl-3-trimethylammonium-propane (DMTAP), 1,2-dioctadecanoyl-3-trimethylammonium-propane (DOTAP) and DOTAP derivatives such as 1,2-di-(9Z-octadecenoyl)-3-trimethylammonium-propane and 1,2-dihexadecanoyl-3-trimethylammonium-propane, 2-di-(9Z-octadecenoyl)-3-dimethylammonium-propane (DODAP) and DODAP derivatives such as 1,2-ditetradecanoyl-3-dimethylammonium-propane, 1,2-dihexadecanoyl-3-dimethylammonium-propane, and 1,2-dioctadecanoyl-3-dimethylammonium-propane, 1,2-di-0-octadecenyl-3-trimethylammonium propane (DOTMA), 1,2-dioleoyl-c-(4′-trimethylammonium)-butanoyl-sn-glycerol (DOTB), dioctadecylamide-glycylspermine, SAINT-2 and 1-palmitoyl-2-oleoyl-sn-glycero-3-ethylphosphocholine (EPC).

Examples of divalent cationic lipids include DiArginine (GWRR) with a lipid anchor carrying a divalent cationic charge (DiArg) An example of a trivalent cationic lipid include TriArginine (GWRRR) with a lipid anchor carrying a trivalent cationic charge (TriArg).

Examples of multivalent cationic lipids include lipid polyarginine conjugates, lipid polylysine conjugates, lipid TAT conjugates and lipid chitosan conjugates.

An embodiment of the present disclosure is a cationic liposome, wherein the cationic lipids are selected from the group consisting of stearylamine (SA), lauryltrimethylammonium bromide; cetyltrimethylammonium bromide, myristyltrimethylammonium bromide, dimethyldioctadecylammonium bromide (DDAB), 3β-[N-(N′,N′-dimethylaminoethane)-carbamoyl]cholesterol (DC-Cholesterol), 1,2-ditetradecanoyl-3-trimethylammonium-propane (DMTAP), 1,2-dioctadecanoyl-3-trimethylammonium-propane (DOTAP) and DOTAP derivatives such as 1,2-di-(9Z-octadecenoyl)-3-trimethylammonium-propane and 1,2-dihexadecanoyl-3-trimethylammonium-propane, 1,2-di-(9Z-octadecenoyl)-3-dimethylammonium-propane (DODAP) and DODAP derivatives such as 1,2-ditetradecanoyl-3-dimethylammonium-propane, 1,2-dihexadecanoyl-3-dimethylammonium-propane, and 1,2-dioctadecanoyl-3-dimethylammonium-propane, 1,2-di-0-octadecenyl-3-trimethylammonium propane (DOTMA), 1,2-dioleoyl-c-(4′-trimethylammonium)-butanoyl-sn-glycerol (DOTB), dioctadecylamide-glycylspermine, SAINT-2, polycationic lipid 2,3-dioleyloxy-N-[2(spermine-carboxamido)ethyl]-N,N-dimethyl-1-propanaminiumtrifluoroacetate (DOSPA), 1-palmitoyl-2-oleoyl-sn-glycero-3-ethylphosphocholine (EPC) and GL67™.

A particular embodiment of the present disclosure is a cationic liposome, wherein the cationic lipids are selected from the group consisting of stearylamine (SA), 1,2-dioctadecanoyl-3-trimethylammonium-propane (DOTAP), 1-palmitoyl-2-oleoyl-sn-glycero-3-ethylphosphocholine (EPC) and 1,2-di-(9Z-octadecenoyl)-3-dimethylammonium-propane (DODAP), preferably 1,2-dioctadecanoyl-3-trimethylammonium-propane (DOTAP).

Further examples of cationic lipids include cationic lipopeptide selected from the group consisting of lipid polyarginine conjugate, TriArginine (GWRRR) with a lipid anchor carrying a trivalent cationic charge (TriArg), a lipid TAT conjugate, a lipid polylysine conjugate, or a cationic liposaccharide or lipopolysaccharide such as a lipid chitosan conjugate.

Preferred cationic lipids of the present disclosure are 1,2-dioctadecanoyl-3-trimethylammonium-propane (DOTAP) and DOTAP derivatives.

Additional examples of cationic lipids and lipid components may be found in or made according to U.S. Pat. No. 4,804,539, the teachings of which are incorporated by reference.

The cationic liposomes of the present disclosure comprise about 0.1-30 mol % of a cationic lipid, wherein the content of cationic lipid is selected such that the cationic lipid content in mol % multiplied by the charge of the cationic lipid is in the range of about 15-30, more preferably at least 16, for example at least 17, such as at least 18, for example at least 19, such as least 20.

As an example, the content of a monovalent cationic lipid in the range of about (15-30 mol %)/1, equals about 15-30 mol %, as an example, the content of a divalent cationic lipid in the range of about (15-30 mol %)/2, equals about 7.5-15 mol %, as an example, the content of a trivalent cationic lipid in the range of about (15-30 mol %)/3, equals about 5-10 mol %, and as an example, the content of a pentavalent lipid in the range of about (15-30 mol %)/5, equals about 3-6 mol %.

An embodiment of the present disclosure is a cationic liposome, wherein the cationic liposomes comprise about 0.1-30 mol %, of a cationic lipid, such that the content of cationic lipid is such that the cationic lipid content in mol % multiplied by the charge of the cationic lipid is in the range of about 16-30, such as about 17-25, for example about 18-22, for example about 23-27, preferably about 20-25.

An embodiment of the present disclosure is a cationic liposome, wherein the cationic liposomes comprise about 0.1-30 mol %, of a cationic lipid, such that the content of cationic lipid is selected such that the cationic lipid content in mol % multiplied by the charge of the cationic lipid is in the range of about 15-25, more preferably at least 16, for example at least 17, such as at least 18 for example at least 19, such as least 20.

As an example, the content of a monovalent cationic lipid is in the range of about (16-30 mol %)/1, equals about 16-30 mol %, as an example, the content of a divalent cationic lipid is in the range of about (16-30 mol %)/2, equals about 8-15 mol %, as an example, the content of a trivalent cationic lipid is in the range of about (16-30 mol %)/3, equals about 5.33-10 mol %, and as an example, the content of a pentavalent lipid is in the range of about (16-30 mol %)/5, equals about 3.2-6 mol %.

An embodiment of the present disclosure is a cationic liposome, wherein the cationic liposomes comprise about 0.1-30 mol %, of a cationic lipid, such that the content of cationic lipid is selected such that the cationic lipid content in mol % multiplied by the charge of the cationic lipid is in the range of about 16-30, for example about 16-25, for example about 18-22, for example about 23-27, preferably about 20-25.

An embodiment of the present disclosure is a cationic liposome, wherein the cationic liposomes comprise about 0.1-30 mol %, of a cationic lipid, such that the content of cationic lipid is selected such that the cationic lipid content in mol % multiplied by the charge of the cationic lipid is in the range of about 16-25.

As it appears from the results reported herein, at ranges of cationic lipids in the above range superior selectivity for monocytes and dendritic cells is obtained.

### PEG

The liposomes of the present disclosure comprise PEG. The PEG is in the form of PEG conjugated to a phospholipid and is present in the range of about 1-10 mol %. The size of the PEG is between PEG350 to PEG30.000.

In one embodiment, the cationic liposome comprises PEG in the range of 1-10 mol %, such as in the range of 3-7 mol %.

The size and content of PEG conjugated to a phospholipid for the specific cationic liposome is usually selected so that the zeta potential is in the range of about 13-25 mV.

In a preferred embodiment, the cationic liposome comprises about 5 mol % PEG2000 conjugated to a phospholipid.

In one embodiment, the size of the PEG is between PEG350 and PEG5000, for example between PEG750 and PEG5000, for example between PEG1000 and PEG5000, for example between PEG2000 and PEG5000, for example between PEG3000 and PEG5000, preferably the size of the PEG is PEG2000.

In one embodiment, the size of the PEG is between PEG350 and PEG5000, for example between PEG350 and PEG3000, for example between PEG350 and PEG2000, for example between PEG350 and PEG1000, for example between PEG350 and PEG750, preferably the size of the PEG is PEG2000.

In one embodiment, the size of the PEG is between PEG350 and PEG5000, for example between PEG550 and PEG4000, for example between PEG750 and PEG3000, such as between PEG1000 and PEG3000, preferably the size of the PEG is PEG2000.

In one embodiment, the phospholipid conjugated to the PEG is selected from the group consisting of 1,2-Dioleoyl-sn-glycero-3-phosphoethanolamine (DOPE), Cholesterol, 1,2-Distearoyl-sn-glycero-3-phosphoethanolamine (DSPE), 1,2-Dimyristoyl-sn-glycero-3-phosphoethanolamine (DMPE), Ceramide and 1,2-Dipalmitoyl-sn-glycero-3-phosphoethanolamine (DPPE), preferably the phospholipid conjugated to the PEG is 1,2-Dioleoyl-sn-glycero-3-phosphoethanolamine (DOPE).

In a preferred embodiment, the PEG is PEG2000 conjugated to 1,2-Dioleoyl-sn-glycero-3-phosphoethanolamine (DOPE), i.e. DOPE-PEG2000.

In a preferred embodiment, PEG is positioned at both the interior surface and the exterior surface of the cationic liposome.

### Cholesterol

The cationic liposomes of the present disclosure may comprise cholesterol in the range of about 0-40 mol %.

In one embodiment, the content of cholesterol is in the range of about 0-40 mol %, for example about 10-40 mol %, for example about 20-40 mol %, such as about 30-40 mol %, preferably the content of cholesterol is about 30 mol %.

In one embodiment, the content of cholesterol is in the range of about 0-40 mol %, for example about 0-30 mol %, for example about 0-20 mol %, such as about 0-10 mol %, preferably the content of cholesterol is about 30 mol %.

In one embodiment, the content of cholesterol is in the range of about 0-40 mol %, for example about 10-40 mol %, for example about 25-35 mol %, preferably the content of cholesterol is about 30 mol %.

In one embodiment, the content of cholesterol is in the range of about 25-35 mol %.

### Phospholipid

The cationic liposomes described herein comprise phospholipids and/or sterol derivatives.

A particular embodiment of the present disclosure is a cationic liposome, wherein the lipid comprises or constitutes a member selected from the group consisting of phosphatidylcholine (PC), phosphatidylethanolamine (PE), phosphatidylserine (PS), phosphatidylglycerol (PG), phosphatidylinositol (PI), phosphatidic acid (PA), DPG (bisphosphatidyl glycerol), PEOH (phosphatidyl alcohol) ergosterol and lanosterol.

An embodiment of the present disclosure is a cationic liposome, wherein the phosphatidylcholines are selected from the group consisting of 1,2-dioleoyl-phosphatidylcholine, 1,2-dipalmitoyl-phosphatidylcholine, 1,2-dimyristoyl-phosphatidylcholine, 1,2-distearoyl-phosphatidylcholine, 1-oleoyl-2-palmitoyl-phosphatidylcholine, 1-oleoyl-2-stearoyl-phosphatidylcholine, 1-palmitoyl-2-oleoyl-phosphatidylcholine and 1-stearoyl-2-oleoyl-phosphatidylcholine.

A preferred embodiment of the present disclosure is a cationic liposome, wherein the phosphatidylcholines is 1-palmitoyl-2-oleoyl-phosphatidylcholine (POPC).

An embodiment of the present disclosure is a cationic liposome, which, when relevant, contains alkyl chains of the phospholipids that are C8-C24, preferably C10-C22, more preferred C12-C20, preferably C14-C18, most preferred C16-C18 saturated chains or unsaturated chains, preferably unsaturated chains.

The content of phospholipid in the cationic liposomes of the present disclosure is selected so that the total content of the cationic liposome is 100 mol %.

As an example, the phospholipid content of a cationic liposome comprising
a) 30 mol % cholesterol,
b) 25 mol % DOTAP,
c) 5 mol % DOPE-PEG2000, and
d) 5 mol % 1v270

is (100 mol %−(30 mol %+25 mol %+5 mol %+5 mol %))=35 mol %.

### Liposome pH

In one embodiment, the pH of the interior cavity of the cationic liposome is in the range of about 5-9, for example in the range of about 5-8, for example in the range of about 6-8.

### Liposome Size

An embodiment of the present disclosure is a cationic liposome, wherein the cationic liposomes have a diameter of about 50-500 nm, for example about 70-200 nm, for example about 80-150 nm, preferably the diameter of the cationic liposomes is in the range of about 115-150 nm.

An embodiment of the present disclosure is a cationic liposome, wherein at least one cationic liposome is a Large Unilamellar Vesicle (LUV).

An embodiment of the present disclosure is a cationic liposome, wherein at least one cationic liposome is a Multilamellar Vesicle (MLV).

### Pharmaceutical Compositions of the Present Disclosure

The cationic liposomes of the present disclosure are useful as constituents of a pharmaceutical formulation. Thus, in one embodiment, the present disclosure provides a pharmaceutical composition comprising the cationic liposome as described herein.

Any form of such formulation which is suitable for administration to a mammal is contemplated.

The pharmaceutical formulation according to the present disclosure is preferably in the form of a solution, dispersion, suspension, lyophilisate, or frozen form.

In one embodiment, the administration route may be intravenous, oral, subcutaneous, intradermal, intramuscular, nasal, intraperitoneal, pulmonary or renal administration.

### Therapeutic Uses or Methods

The cationic liposome of the present disclosure may be used in prophylaxis, treatment or amelioration of cancer, an infectious disease, an inflammatory condition or disease, an autoimmune disease or allergy.

In one embodiment, the cationic liposome is used in prophylaxis, treatment or amelioration of cancer.

In one embodiment, the cationic liposome is used in prophylaxis, treatment or amelioration of an infectious disease.

In certain embodiments, the cationic liposomes of the present disclosure are used to deliver active ingredients to monocytes in vitro or ex vivo. In such settings, fresh blood is drawn from a patient in need of such treatment, the liposomes are used to deliver the active ingredient specifically to the patient's monocytes or dendritic cells in vitro or in ex vivo conditions in isolated conditions for a period of time and the monocytes are re-introduced into the patient.

An embodiment of the present disclosure is a method for in vitro activation or inhibition of monocytes and dendritic cells, comprising the steps of (i) providing fresh blood, (ii) administering the cationic liposome to said fresh blood, and (iii) allowing said cationic liposome, lipid-based delivery system or pharmaceutical composition to react.

An embodiment of the present disclosure is a method for in vivo activation or inhibition of monocytes and dendritic cells in a subject, comprising administering the cationic liposome to said subject in an amount sufficient to activate or inhibit said monocytes and dendritic cells.

An embodiment of the present disclosure is a method for ex vivo activation or inhibition of monocytes and dendritic cells, comprising the steps: (i) providing fresh blood from a subject, (ii) administering the cationic liposome to said fresh blood, (iii) allowing said cationic liposome, lipid delivery system or pharmaceutical composition to react with said fresh blood, and (iv) reintroducing said blood into the circulation of said subject.

An embodiment of the present disclosure is a method for prophylactic or therapeutic treatment or amelioration of cancer, an infectious disease, an inflammatory condition or disease, an autoimmune disease or allergy, the method comprising administering to a subject in need thereof an effective amount of the cationic liposome as described herein.

### Items

Further details of the present disclosure are provided in the following items.
1. A cationic liposome comprising:
   a. between 0-40 mol % cholesterol,
   b. between 1-10 mol % PEG conjugated to a phospholipid,
   c. at least one cationic lipid, and
   d. at least one active ingredient,
   wherein the remaining components are phospholipids and wherein the zeta potential is in the range of 13-25 mV.
2. The cationic liposome according to item 1, wherein the active ingredient is an immunomodulatory agent.
3. The cationic liposome according to item 2, wherein the active ingredient is an immunostimulating compound which is a ligand for an intracellular protein and/or receptor selected from the group consisting of STING, TLR3, TLR7, TLR8, TLR9, NOD1, NOD2, NOD5, NALP1, NALP2, NALP3, NALP12, NALP14, IPAF, NAIP, CIITA, RIG-I, MDA5, and LGP2, preferably selected from STING, TLR3, TLR7, TLR8, TLR9, and NOD2, more preferably TLR7.
4. The cationic liposome according to item 2, wherein the active ingredient is a TLR7 agonist, such as a TLR7 agonist selected from the group consisting of Formula (I), Formula (II), Formula (III) and Formula (IV).

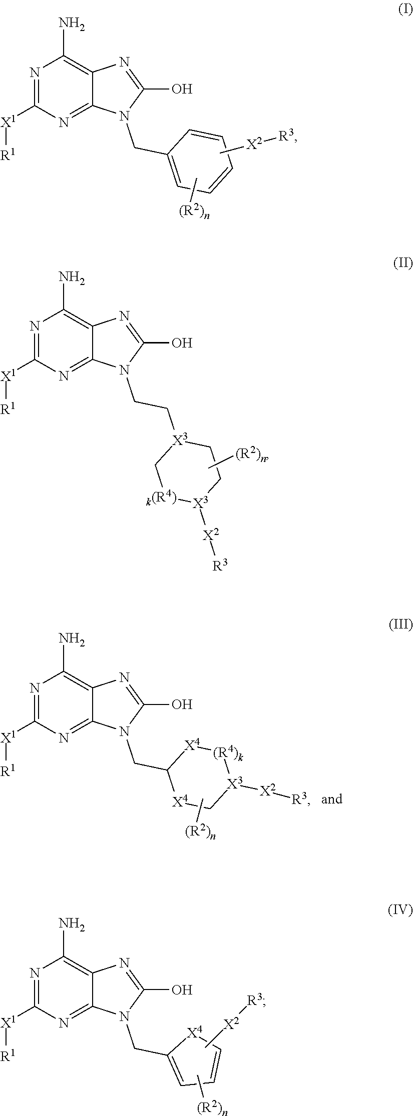

wherein X¹ is -0-, —S—, or —NR^{C};
R¹ is hydrogen, (C₁-C₁₀)alkyl, substituted (C₁-C₁₀)alkyl, C₆₋₁₀aryl, or substituted C₆₋₁₀aryl, C₅₋₉heterocyclic, substituted C₅₋₉heterocyclic;
R^{C} is hydrogen, C₁₋₁₀alkyl, or substituted C₁₋₁₀alkyl; or R^{C} and R¹ taken together with the nitrogen to which they are attached form a heterocyclic ring or a substituted heterocycli ring;
each R² is independently —OH, (C₁-C₆)alkyl, substituted (C₁-C₆)alkyl, (C₁-C₆)alkoxy, substituted (C₁-C₆)alkoxy, —C(0)-(C₁-C₆)alkyl (alkanoyl), substituted —C(0)-(C₁-C₆)alkyl, —C(0)-(C₆-C₁₀)aryl (aroyl), substituted —C(O)—(C₆-C₁₀)aryl, —C(0)OH (carboxyl), —C(0)0(C₁-C₆)alkyl (alkoxycarbonyl), substituted —C(0)0(C₁-C₆)alkyl, —NR^{a}R^{b}, —C(O)NR^{a}R^{b} (carbamoyl), halo, nitro, or cyano, or R² is absent; each R^{a} and R^{b} is independently hydrogen, (C₁-C₆)alkyl, substituted (C₁-C₆)alkyl, (C₃-C₈)cycloalkyl, substituted (C₃-C₈)cycloalkyl, (C₁-C₆)alkoxy, substituted (C₁-C₆)alkoxy, (C₁-C₆)alkanoyl, substituted (C₁-C₆)alkanoyl, aryl, aryl(C₁-C₆)alkyl, Het, Het (C₁-C₆)alkyl, or (C₁-C₆)alkoxycarbonyl;
wherein the substituents on any alkyl, aryl or heterocyclic groups are hydroxy, C₁₋₆alkyl, hydroxyC₁₋₆alkylene, C₁₋₆alkoxy, C₃₋₆cycloalkyl, C₁₋₆alkoxy C₁₋₆alkylene, amino, cyano, halo, or aryl;
n is 0, 1, 2, 3 or 4;
X² is a bond or a linking group; and
R³ is a phospholipid comprising one or two carboxylic esters;
X³ is —N— or —CH—;
R⁴ is —CH2- or —CH(R<2>)-; and
k is 0 or 1;
X⁴ is -0-, —S—, —NH—, —N(R^{d})—, —CH₂—, or —CH(R²)—;
each R^{d} is independently —OH, (C₁-C₆)alkyl, substituted (C₁-C₆)alkyl, (C₁-C₆)alkoxy, substituted (C₁-C₆)alkoxy, —C(0)-(C₁-C₆)alkyl (alkanoyl), substituted —C(0)-(C₁-C₆)alkyl, —C(0)-(C₆-C₁₀)aryl (aroyl), substituted —C(O)—(C₆-C₁₀)aryl, —C(0)0(C₁-C₆)alkyl (alkoxycarbonyl), substituted —C(0)0(C₁-C₆)alkyl, —C(0)NR^{a}R^{b} (carbamoyl);
or a tautomer thereof;
or a pharmaceutically acceptable salt or solvate thereof, and
wherein the ring system of formula (II) is a piperidin ring with one heteroatom being an N atom and with the N-atom of the piperidin ring adjacent to X², and wherein the purine group in any of Formula (I), (II), (III), or (IV) is subject to tautomeric rearrangements.
5. The cationic liposome according to item 4, wherein the TLR7 agonist has a structure according to Formula (IA).

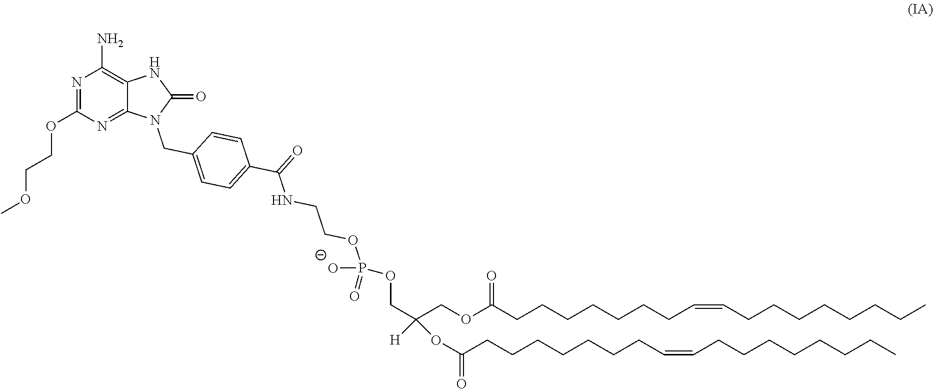 [SMILES: CCCCCCCC/C=C\CCCCCCCC(=O)OCC(COP(=O)([O-])OCCNC(=O)c1ccc(Cn2c(=O)[nH]c3c(N)nc(OCCOC)nc32)cc1)OC(=O)CCCCCCC/C=C\CCCCCCCC]

6. The cationic liposome according to item 2, wherein the immunomodulatory agent is an immunosuppressive compound.
7. The cationic liposome according to any of the preceding items, wherein the content of the active ingredient is in the range of about 0.1-50 mol %, for example about 2-40 mol %, for example about 5-30 mol %, for example about 10-20 mol %, for example about 2.5-7.5 mol %, such as about 3-7 mol %, preferably the content of the active ingredient is about 5 mol %.
8. The cationic liposome according to any of the preceding items, wherein the zeta potential is about 15-25 mV.
9. The cationic liposome according to any of the preceding items, wherein the cationic lipid is selected from monovalent cationic lipids, divalent cationic lipids, trivalent cationic lipids or multivalent cationic lipids.
10. The cationic liposome according to any of the preceding items, wherein the cationic lipid is selected from the group consisting of stearylamine (SA), lauryltrimethylammonium bromide; cetyltrimethyl-ammonium bromide, myristyl trimethylammonium bromide, dimethyldioctadecylammonium bromide (DDAB), 36-[N-(N′,N′-dimethylaminoethane)-carbamoyl]cholesterol (DC-Cholesterol), 1,2-ditetradecanoyl-3-trimethylammonium-propane (DMTAP), 1,2-dioctadecanoyl-3-trimethylammonium-propane (DOTAP) and DOTAP derivatives such as 1,2-di-(9Z-octadecenoyl)-3-trimethylammonium-propane and 1,2-dihexadecanoyl-3-trimethylammonium-propane, 1,2-di-(9Z-octadecenoyl)-3-dimethylammonium-propane (DODAP) and DODAP derivatives such as 1,2-ditetradecanoyl-3-dimethylammonium-propane, 1,2-dihexadecanoyl-3-dimethylammonium-propane, and 1,2-dioctadecanoyl-3-dimethylammonium-propane, 1,2-di-0-octadecenyl-3-trimethylammonium propane (DOTMA), 1,2-dioleoyl-c-(4′-trimethylammonium)-butanoyl-sn-glycerol (DOTB), dioctadecylamide-glycylspermine, SAINT-2, polycationic lipid 2,3-dioleyloxy-N-[2(spermine-carboxamido)ethyl]-N,N-dimethyl-1-propanaminiumtrifluoroacetate (DOSPA), 1-palmitoyl-2-oleoyl-sn-glycero-3-ethylphosphocholine (EPC) and GL67™.
11. The cationic liposome according to any of the preceding items, wherein the cationic lipid is a cationic lipopeptide selected from the group consisting of a lipid polyarginine conjugate, TriArginine (GWRRR) with a lipid anchor carrying a trivalent cationic charge (TriArg), a lipid TAT conjugate, a lipid polylysine conjugate, or a cationic liposaccharide or lipopolysaccharide such as a lipid chitosan conjugate.
12. The cationic liposome according to any of the preceding items, wherein the cationic lipid is 1,2-dioctadecanoyl-3-trimethylammonium-propane (DOTAP).
13. The cationic liposome according to any of the preceding items wherein the content of cationic lipid in mol % multiplied by the charge of the cationic lipid is in the range of 16-30, such as 20-30, such as 20-25.
14. The cationic liposome according to any of the preceding items, wherein the cationic lipid is a monovalent cationic lipid and the content of the monovalent cationic lipid is in the range of about 16-30 mol %, such as 20-30 mol %.
15. The cationic liposome according to any of the preceding items, wherein the cationic lipid is a divalent cationic lipid and the content of the divalent cationic lipid is in the range of about 8-15 mol %, such as 10-15 mol %.
16. The cationic liposome according to any of the preceding items, wherein the cationic lipid is a trivalent cationic lipid and the content of the trivalent cationic lipid is in the range of about 5.5-10 mol %, such 6-10 mol %, such as 7-10 mol %, such as 8-10 mol %.
17. The cationic liposome according to any of the preceding items, wherein the cationic lipid is a multivalent cationic lipid and the content of the multivalent cationic lipid is in the range of about 16-30 mol % divided by the charge of the lipid.
18. The cationic liposome according to any of the preceding items, wherein the size of the PEG is between PEG350 and PEG5000, for example between PEG550 and PEG4000, for example between PEG750 and PEG3000, such as between PEG1000 and PEG3000, preferably the size of the PEG is PEG2000.
19. The cationic liposome according to any of the preceding items, wherein the size and content of PEG conjugated to a phospholipid for the specific cationic liposome is selected so that the zeta potential of the cationic liposome is in the range of about 13-25 mV.
20. The cationic liposome according to any of the preceding items, wherein the phospholipid conjugated to PEG is selected from the group consisting of, 1,2-Dioleoyl-sn-glycero-3-phosphoethanolamine (DOPE), Cholesterol, 1,2-Distearoyl-sn-glycero-3-phosphoethanolamine (DSPE), 1,2-Dimyristoyl-sn-glycero-3-phosphoethanolamine (DMPE), Ceramide and 1,2-Dipalmitoyl-sn-glycero-3-phosphoethanolamine (DPPE), preferably the phospholipid conjugated to the PEG is 1,2-Dioleoyl-sn-glycero-3-phosphoethanolamine (DOPE).
21. The cationic liposome according to any of the preceding items, wherein the PEG conjugated to a phospholipid is PEG2000 conjugated to 1,2-Dioleoyl-sn-glycero-3-phosphoethanolamine (DOPE).
22. The cationic liposome according to any of the preceding items, wherein the content of cholesterol is in the range of about 0-40 mol %, for example about 10-40 mol %, for example about 25-35 mol %, preferably the content of cholesterol is about 30 mol %.
23. The cationic liposome according to any of the preceding items, wherein the phospholipid is selected from the group consisting of phosphatidylcholine (PC), phosphatidylethanolamine (PE), phosphatidylserine (PS), phosphatidylglycerol (PG), phosphatidylinositol (PI), phosphatidic acid (PA), DPG (bisphosphatidyl glycerol), PEOH (phosphatidyl alcohol), ergosterol and lanosterol, preferably the phospholipid is a phosphatidylcholine.
24. The cationic liposome according to item 23, wherein the phosphatidylcholine is selected from the group consisting of 1,2-dioleoyl-phosphatidylcholine, 1,2-dipalmitoyl-phosphatidylcholine, 1,2-dimyristoyl-phosphatidylcholine, 1,2-distearoyl-phosphatidylcholine, 1-oleoyl-2-palmitoyl-phosphatidylcholine, 1-oleoyl-2-stearoyl-phosphatidylcholine, 1-palmitoyl-2-oleoyl-phosphatidylcholine and 1-stearoyl-2-oleoyl-phosphatidylcholine, preferably the phosphatidylcholine is 1-palmitoyl-2-oleoyl-phosphatidylcholine (POPC).
25. The cationic liposome according to any of the preceding items, wherein the pH of the interior cavity of the cationic liposome is in the range of about 5-9, for example in the range of about 5-8, for example in the range of about 6-8.
26. The cationic liposome according to any of the preceding items, wherein the at least one active ingredient comprises at least two immunomodulatory agents.
27. The cationic liposome according to any of the preceding items, wherein the at least one active ingredient comprises at least one immunomodulatory agent and at least one antigen.
28. The cationic liposome according to item 27, wherein the at least one antigen is selected from the group consisting of a cancer antigen, a self- or autoimmune antigen, a microbial antigen, an allergen, or an environmental antigen.
29. The cationic liposome according to any of the preceding items, wherein the cationic liposomes have a diameter of about 50-500 nm, for example about 70-200 nm, for example about 80-150 nm, preferably the diameter of the cationic liposomes is in the range of about 115-150 nm.
30. A cationic liposome comprising:
   a. between 25-35 mol % cholesterol,
   b. between 3-7 mol % PEG conjugated to a phospholipid,
   c. at least one cationic lipid, and
   d. at least one active ingredient,
   wherein the remaining components are phospholipids and wherein the zeta potential is in the range of 15-25 mV.
31. A cationic liposome comprising:
   a. between about 30-40 mol % POPC, such as about 35 mol % POPC,
   b. between about 25-35 mol % cholesterol, such as about 30 mol % cholesterol,
   c. between about 3-7 mol % DOPE-PEG2000, such as about 5 mol % DOPE-PEG2000,
   d. between about 20-30 mol % DOTAP, such as about 25 mol % DOTAP, and
   e. between about 2.5-7.5 mol % of the TLR7 agonist 1v270, such as about 5 mol % 1v270,
   wherein the zeta potential is in the range of 13-25 mV.
32. A cationic liposome comprising:
   a. between about 50-60 mol % POPC, such as about 54 mol % POPC,
   b. between about 25-35 mol % cholesterol, such as about 30 mol % cholesterol,
   c. between about 3-7 mol % DOPE-PEG2000, such as about 5 mol % DOPE-PEG2000,
   d. between about 4-8 mol % TriArg, such as about 6 mol % TriArg, and
   e. between about 2.5-7.5 mol % of the active ingredient, such as a TLR7 agonist,
   wherein the zeta potential is in the range of 13-25 mV.
33. A cationic liposome comprising:
   a. between about 35-45 mol % POPC, such as about 40 mol % POPC
   b. between about 25-35 mol % cholesterol, such as about 30 mol % cholesterol,
   c. between about 3-7 mol % DOPE-PEG2000, such as about 5 mol % DOPE-PEG2000,
   d. between about 15-25 mol % EPC, such as about 20 mol % EPC, and
   e. between about 2.5-7.5 mol % of the active ingredient, such as a TLR7 agonist,
   wherein the zeta potential is in the range of 13-25 mV.
34. The cationic liposome according to any of the preceding items, wherein the cationic liposome preferentially adheres to monocytes and dendritic cells in fresh whole blood when compared to adherence to granulocytes, T-lymphocytes, B-lymphocytes and/or NK cells.
35. The cationic liposome according to any of the preceding items, wherein the cationic liposome provides delivery to and release of the at least one active ingredient to a target cell, preferably a monocyte and dendritic cells.
36. A lipid-based delivery system for targeting monocytes and dendritic cells in fresh whole blood, said system providing delivery to and release of at least one active ingredient to the targeted monocyte, and said system comprising:
   a. between 0-40 mol % cholesterol,
   b. between 2-10 mol % PEG conjugated to a phospholipid,
   c. at least one cationic lipid, and
   d. at least one active ingredient;
   wherein the remaining components are phospholipids and wherein the zeta potential is in the range of 13-25 mV,
   said system allowing the formation of cationic liposomes, at which said cationic liposomes preferentially adheres to monocytes and dendritic cells in fresh whole blood when compared to adherence to granulocytes, T-lymphocytes, B-lymphocytes and/or NK cells.
37. A pharmaceutical composition comprising the cationic liposome according to any one of claims 1-36.
38. The cationic liposome according to any one of items 1-31, the lipid-based delivery system according to item 36 or the pharmaceutical composition according to item 37, for use as a pharmaceutical.
39. The cationic liposome according to any one of items 1-31, the lipid-based delivery system according to item 36 or the pharmaceutical composition according to item 37, for use in prophylaxis, treatment or amelioration of cancer, an infectious disease, an inflammatory condition or disease, an autoimmune disease or allergy.
40. Use of the cationic liposome according to any one of items 1-31, the lipid-based delivery system according to item 36 or the pharmaceutical composition according to item 37, for the manufacture of a medicament for prophylaxis, treatment or amelioration of cancer, an infectious disease, an inflammatory condition or disease, an autoimmune disease or allergy.
41. A method for in vitro activation or inhibition of monocytes and dendritic cells, comprising the steps of:
   i) providing fresh blood,
   ii) administering a cationic liposome according to any one of items 1-31, the lipid-based delivery system according to item 36 or the pharmaceutical composition according to item 37 to said fresh blood, and
   iii) allowing said cationic liposome, lipid-based delivery system or pharmaceutical composition to react.
42. A method for in vivo activation or inhibition of monocytes and dendritic cells in a subject, comprising administering the cationic liposome according to any one of items 1-31, the lipid-based delivery system according to item 36 or the pharmaceutical composition according to item 37 to said subject in an amount sufficient to activate or inhibit said monocytes and dendritic cells.
43. A method for ex vivo activation or inhibition of monocytes and dendritic cells, comprising the steps:
   i) providing fresh blood from a subject,
   ii) administering the cationic liposome according to any one of items 1-31, the lipid-based delivery system according to item 36 or the pharmaceutical composition according to item 37 to said fresh blood,
   iii) allowing said cationic liposome, lipid delivery system or pharmaceutical composition to react with said fresh blood, and
   iv) reintroducing said blood into the circulation of said subject.
44. A method for ex vivo activation or inhibition of monocytes and dendritic cells, comprising the steps:
   i) administering the cationic liposome according to any one of items 1-31, the lipid-based delivery system according to item 36 or the pharmaceutical composition according to item 37 to a fresh blood sample obtained from a subject,
   ii) allowing said cationic liposome, lipid delivery system or pharmaceutical composition to react with said fresh blood sample.
45. A method for prophylactic or therapeutic treatment or amelioration of cancer, an infectious disease, an inflammatory condition or disease, an autoimmune disease or allergy, the method comprising administering to a subject in need thereof an effective amount of the cationic liposome according to any one of items 1-31, the lipid-based delivery system according to item 36 or the pharmaceutical composition according to item 37.

### EXAMPLES

### Example 1: Liposome Preparation

Unilamellar fully hydrated liposomes were made from mixtures of 1-palmitoyl-2-oleoyl-sn-glycero-3-phosphocholine (POPC), 1,2-dioleoyl-sn-glycero-3-phosphoethanolamine-N-[methoxy(polyethylene glycol)-2000] (DOPE-PEG2000), cholesterol (CHOL) and a cationic component. One of three cationic lipids were used (1,2-dioleoyl-3-trimethylammonium-propane (DOTAP), TriArginine (GWRRR) with a lipid anchor carrying a trivalent cationic charge (TriArg) and 1-palmitoyl-2-oleoyl-sn-glycero-3-ethylphosphocholine (EPC). As a fluorescence marker to measure presence of liposomes in biological systems, 0.1% 1-palmitoyl-2-(dipyrrometheneboron difluoride) undecanoyl-sn-glycero-3-phosphocholine (TopFI) or 0.05% 1,2-dioleoyl-sn-glycero-3-phosphoethanolamine-Atto488 (Atto488) was mixed with the lipids as a tracer. The molar ratios of each lipid in the liposomes are outlined in Table 1. All lipids were obtained from Avanti Polar lipids or Lipoid, except for Atto488 which was obtained from AttoTec GmbH.

TABLE 1
Overview of cationic liposomes used, liposome name, composition, molar ratio, surface charge (Zeta), size and Polydispersity Index (PDI).
Liposomes were composed of 1-palmitoyl-2-oleoyl-sn-glycero-3-phosphocholine (POPC), Cholesterol (Chol) and 1,2-dioleoyl-sn-glycero-
3-phosphoethanolamine-N-[methoxy(polyethylene glycol)-2000] (DOPE-PEG2000 or PEG), TLR7 agonist 1v270 (2-(4-((6-amino-2-(2-methoxyethoxy)-
8-oxo-7H-purin-9(8H)-yl)methyl)benzamido)ethyl 2,3-bis(oleoyloxy)propyl phosphate) (1v270), 1-palmitoyl-2-(dipyrrometheneboron difluoride)
undecanoyl-sn-glycero-3-phosphocholine (TopFl), 1,2-dioleoyl-sn-glycero-3-phosphoethanolamine-Atto488 (Atto488), Tri-Arginine lipid
derivative (TriArg), 1-palmitoyl-2-oleoyl-sn-glycero-3-ethylphosphocholine (EPC). Composition and molar ratio of each component in
the liposomes is shown. Liposome surface charge is expressed in mV. Liposome size is measured in nanometer (nm). Error is expressed
as SEM. Liposome names from Table 1 are used throughout in the figures and examples to clarify exact composition.

| Name | Application | Composition | Molar ratio | Zeta (mV) | Size (nm) | PDI |
|---|---|---|---|---|---|---|
| Cationic-PEG-1v270 Lip | Ex vivo | POPC:Chol:DOTAP:DOPE- | 34.9:30:25:5:5:0.1 | 15.8 | 138.0 | 0.046 |
| | experiments | PEG2000:1v270:TopFl | | | | |
| | for flow | | | | | |
| | In vivo | POPC:CHOL:DOTAP:1v270:DOPE-PEG2000 | 35:30:25:5:5 | 14.4 ± 0.4 | 148 | 0.06 |
| | experiments | | | | | |
| Neutral PEG-1V270 Lip | Ex vivo | POPC:Chol:DOTAP:DOPE- | 54.9:30:5:5:5:0.1 | −8.49 ± 2.35 | 124.9 ± 0.9 | 0.035 |
| | experiments | PEG2000:1v270:TopFl | | | | |
| | for flow | | | | | |
| | In vivo | POPC:CHOL:1v270:DOPE-PEG2000 | 60:30:5:5 | −6.4 ± 2.4 | 131 | 0.04 |
| | experiments | | | | | |
| Cationic 1v270 Lip | In vivo | POPC:CHOL:DOTAP:1v270 | 52.5:30:12.5:5 | 32.0 ± 0.3 | 149 | 0.13 |
| | experiments | | | | | |
| Cationic -PEG Lip | Ex vivo | POPC:Chol:DOTAP:DOPE-PEG2000:TopFl | 44.9:30:20:5:0.1 | 22.31 ± 1.00 | 126.9 ± 1.1 | 0.062 |
| | experiments | | | | | |
| | for flow | | | | | |
| | Ex vivo | POPC:Chol:DOTAP:DOPE-PEG2000:TopFl | 44.9:30:20:5:0.1 | 15.46 | 137.2 | 0.036 |
| | experiments | | | | | |
| | for flow | | | | | |
| Cationic TriArg-PEG Lip | 6% TriArg | POPC:Chol:TriArg:DOPE-PEG2000:Atto488 | 58.95:30:6:5:0.05 | 17.83 ± 1.30 | 120.8 ± 1.6 | 0.012 |
| Cationic TriArg Lip | 2% TriArg | POPC:Chol:TriArg:Atto488 | 67.95:30:2:0.05 | 34.3 ± 2.21 | 142.3 ± 0.9 | 0.055 |
| Cationic -PEG Lip | 20 DOTAP-PEG | POPC:Chol:DOTAP:DOPE-PEG2000:Atto488 | 44.95:30:20:5:0.05 | 19.00 ± 1.29 | 127.4 ± 0.9 | 0.088 |
| Cationic Lip | 7.5% DOTAP | POPC:Chol:DOTAP:Atto488 | 62.45:30:7.5:0.05 | 36.61 ± 1.65 | 118.6 ± 1.7 | 0.115 |
| Cationic EPC-PEG Lip | 20% EPC PEG | POPC:Chol:EPC:DOPE-PEG2000:Atto488 | 44.95:30:20:5:0.05 | 18.68 ± 1.32 | 138.8 ± 1.6 | 0.050 |
| Cationic EPC Lip | 7.5% EPC | POPC:Chol:EPC:Atto488 | 62.45:30:7.5:0.05 | 31.34 ± 1.86 | 130.1 ± 0.3 | 0.019 |

Briefly, appropriate weighed amounts of POPC, DOTAP, TriArg, EPC, DOPE-PEG2000, CHOL, Atto488 and TopFI were dissolved in tert-butanol and mixed to the desired lipid ratio in glass vials. The solvent was removed by freezing the vials in liquid nitrogen followed by overnight lyophilization. Multilamellar vesicles were prepared by dispersing the dried lipids in a buffer solution containing: 150 mM NaCl, 10 mM Phosphate (pH=7.0), and placing under magnet stirring for 1 hour. The multilamellar vesicles were extruded 21 times through a 100 nm pore size polycarbonate filters (Whatman) as described by Mayer et al., Biochim. Biophys. Acta, 858, 161-168, using a mini-extruder from Avanti.

### Example 2: Liposome Preparation with Incorporation of 1v270

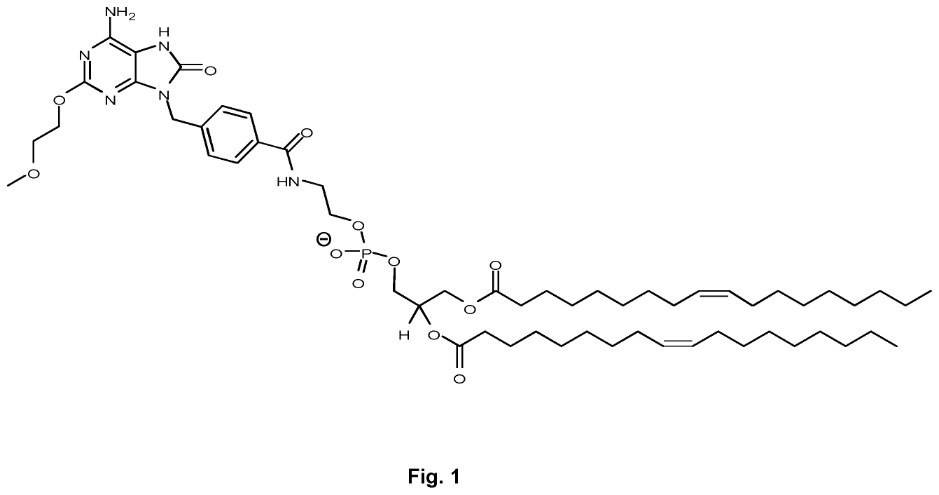

Unilamellar fully hydrated liposomes were made from mixtures of POPC, CHOL, DOTAP and 1v270 (Formula (IA), C₅₇H₉₃N₆O₁₂P, Mw=1085.4, (2-(4-((6-amino-2-(2-methoxyethoxy)-8-oxo-7H-purin-9(8H)-yl)methyl)benzamido)ethyl 2,3-bis(oleoyloxy)propyl phosphate ). The chemical structures of 1v270 is outlined in FIG. 1. As a fluorescence marker to measure presence of liposomes in biological systems, 0.1% 1-palmitoyl-2-(dipyrrometheneboron difluoride) undecanoyl-sn-glycero-3-phosphocholine (TopFI) was mixed with the lipids as a tracer. The molar ratios, lipid concentration, zeta potentials, lipid and 1v270 concentrations are outlined in Table1. The liposomes were prepared as described in Example 1.

### Example 3: Characterization of Liposome Size and Surface Charge Dependent on Composition

Liposomes with compositions as outlined in Table 1 were prepared with the attempt to design liposomes with ability to be recognized and taken up by antigen presenting cells (APCs) like monocytes and dendritic cells but not by other cells in the blood. We designed liposomes with different cationic lipids either with or without DOPE-PEG (Table 1). The liposomes were prepared as described in example 1+2, and their size measured in nanometer (nm) by dynamic light scattering on a ZetaPALS zeta potential analyzer from Brookhaven Instruments in a buffer consisting of 300 mM glucose, 10 mM HEPES, 1 mM CaCl₂ in MilliQ water, pH 7.4. The liposomes showed sizes between 115-150 nm in diameter (Table 1). The surface charge (Zeta potential) of the liposomes was measured in mV and showed surface charge dependent on lipid composition and cationic lipid content. Cationic liposomes with DOPE-PEG showed a typical zeta potential in the range 14-22 mV, and cationic liposomes without DOPE-PEG 2000 showed zeta potential at 30-40 mV, typically at 32 mV (Table 1). Control liposomes were neutral liposomes (Neutral PEG-1v270 Lip) with low amount of cationic lipid content (5%), showed zeta potential at approximately −5 to −10 mV. The same zeta potential was achieved for all three cationic lipids used (TriArg, EPC, DOTAP).

### Example 4: Liposome Targeting to Leukocytes Dependent on Liposome Composition

The cellular uptake of liposome formulations was determined based on fluorescence of TopFI or Atto488 incorporated into the liposomal membrane. The total amount of liposome associated with cells include cell membrane bound liposomes and liposomes already internalized, and was estimated using excitation at 488 and measuring emission at 533/30 nm. Liposomal uptake in monocytes, lymphocytes and granulocytes was analyzed using flow cytometry (Accuri C6 flow cytometer from BD). The following markers were used to distinguish the different populations: CD14 (monocytes), CD19 (B-lymphocytes). The total count of granulocytes and lymphocytes were done based on gating according to morphology using forward and side-scatter (FIG. 2A), CD14 and CD19 staining was gated for within the monocyte and lymphocyte gate respectively. For whole of FIG. 2, control samples represent autofluorescense from non-liposome treated immune cells.

Whole Human Blood was obtained from healthy volunteers under signed consent and collected in hirudin tubes (Roche Diagnostics). The blood was added to tubes containing the liposome stock pre-diluted in RPMI at 500 μM total lipid, and incubated for 1 h at 37° C. with 5% CO₂ under rotation and then washed 3 times in PBS containing 1% FBS. Cells were centrifuged at 200 g for 5 minutes and supernatant discarded. Erythrocytes were lysed in 4 mL BD PharmLyse lysis buffer per 200 μL blood, followed by 15 min incubation in dark at RT. After centrifugation at 200 g for 5 min and removal of supernatant, a second lysis with 1 mL lysis buffer for 5 min was done. Cells were washed twice in cold PBS (1% FBS) to stop the lysis and IgG added (2 μg/10⁶ cells) and incubated on ice for 10 minutes before transferring to a round-bottomed 96 well plate from Nunc (Roskilde, Denmark). 10 μL CD14 specific antibodies pre-labelled with Allophycocyanin was added to stain monocytes and incubated on ice and in dark for 30 minutes. Plate was then spun for 8 min at 400 g and washed with PBS twice. Finally, cells were resuspended in 100 μL PBS. Flow cytometry was performed using an ACCURI C6 flow cytometer from BD, where a minimum of 100000 cells were collected. Fluorescence from the CD14 staining was measured by exciting at 640 nm and measuring at 675/25 nm. Amount of liposome associated with cells was determined using Atto488 emission measured at 533/30 nm with excitation at 488 nm. Analysis was done in the FlowJo software.

Association between cationic liposomes and monocytes, lymphocytes and granulocytes was examined and the Median Fluorescence Intensity (MFI) of these cell populations was analyzed by Flow cytometry (FIG. 2B). The results show first that cationic liposomes are taken up in large amounts in monocytes, and to a very limited extent in granulocytes and only very little association with lymphocytes. Secondly, the data demonstrate that PEGylated liposomes (Cationic TriArg-PEG Lip, Cationic-PEG Lip, Cationic EPC-PEG Lip) show higher uptake to monocytes than non-PEGylated liposomes (Cationic TriArg Lip, Cationic Lip and Cationic EPC Lip). Third, the data show that the increased monocyte uptake is not specific for DOTAP liposomes, but that it is a mechanism dependent on the charge itself, since introduction by a cationic charge by either TriArginine-lipid or EPC show the same monocyte targeting properties (FIG. 2B).

The targeting of PEGylated liposomes (Cationic TriArg-PEG Lip, Cationic-PEG Lip, Cationic EPC-PEG Lip) is shown to be significantly higher than for non-PEGylated liposomes (Cationic TriArg Lip, Cationic Lip and Cationic EPC Lip) towards monocytes when evaluating the MFI value for each type of liposome (FIG. 2C). Looking on the percentage of leukocytes positive for liposome uptake (and not the amount of liposomes taken up as shown by MFI values), monocytes show also superior uptake, with nearly 100% monocytes that have taken up PEGylated liposomes (Cationic TriArg-PEG Lip, Cationic-PEG Lip, Cationic EPC-PEG Lip), and 60-80% of the monocytes have taken up non-PEGylated liposomes (Cationic TriArg Lip, Cationic Lip and Cationic EPC Lip) (FIG. 2D). 15-50% of granulocytes are positive for liposome uptake, whereas only very few lymphocytes take up cationic liposomes (FIG. 2D). Comparing monocyte and granulocytes, the Cationic TriArg-PEG Lip and Cationic-PEG Lip showed a borderline significant or clear tendency to target monocytes more specifically than Cationic TriArg Lip and Cationic Lip (FIG. 2E). Comparing monocyte and lymphocytes as a whole population, the PEGylated liposomes (Cationic TriArg-PEG Lip, Cationic-PEG Lip, Cationic EPC-PEG Lip) are all targeting monocytes significantly more specific than lymphocytes, and with higher specificity than non-PEGylated liposomes (Cationic TriArg Lip, Cationic Lip and Cationic EPC Lip) (FIG. 2F). Comparing monocyte and B-cell uptake, the PEGylated liposomes (Cationic TriArg-PEG Lip, Cationic-PEG Lip, Cationic EPC-PEG Lip) are all targeting monocytes specifically, and significantly better than non-PEGylated liposomes (Cationic TriArg Lip, Cationic Lip and Cationic EPC Lip), which show only a tendency or weakly significant preference for monocytes over B-cells (FIG. 2G).

### Example 5: Cytokine Secretion in Ex Vivo Human Whole Blood Studies

For targeting a liposome to monocytes and antigen presenting cells (APCs) for use in cancer immunotherapy, it is key to boost an appropriate immune response. To analyze this we examined the ability of TLR7 agonist containing liposomes to induce cytokine secretion after targeting as shown in FIG. 2 and example 4, after another 24 h incubation.

Briefly, whole blood from healthy human donors were incubated with liposomes as explained in example 4 and FIG. 2, but after incubation for 1 h with rotation, the samples were washed in RPMI twice, and incubated in 96 well round bottom plates for 24 h. The supernatant was collected and used to measure cytokines by ELISA. For FIG. 4A-D, control samples represent whole blood treated with solvent for liposomes (RPMI-media) or solvent for free 1v270 compound, DMSO diluted as for dilution for 1v270.

IFNα which is secreted mainly from plasmacytoid dendritic cells was significantly induced from the Cationic PEG 1v270 Lip formulation compared to both the free TLR7 agonist 1v270, Neutral PEG 1v270 Lip liposomes which were unable to target monocytes as well as the non-PEGylated Cationic 1v270 Lip. This shows that the Cationic PEG 1v270 Lip formulation is superior in induction of the IFNα compared to the non-PEGylated counterpart Cationic 1v270 Lip (FIG. 3A). IL-12p70 which is secreted mainly from monocytes, monocyte derived DCs (moDC) and myeloid DCs (mDC), and important for boosting an anti-tumor response (Gonzales-Aparicio et al., GUT 2011, 60, 341-349) was significantly induced from the Cationic PEG 1v270 Lip formulation compared to both the free TLR7 agonist 1v270, Neutral PEG 1v270 Lip as well as the non-PEGylated Cationic 1v270 Lip. These findings show that the Cationic PEG 1v270 Lip formulation is superior in induction of IL-12p70 compared to the non-PEGylated counterpart Cationic 1v270 Lip and the free 1v270 TLR7 agonist (FIG. 3B). IL-6 which is secreted from a range of immune cells and is associated with Cytokine Release Syndrome (CRS) and is a critical toxicity parameter for cancer patients treated with certain types of treatment (Lee et al., Blood, 2014, 124(2), 188-195), was analyzed in the same experiments. IL-6 was highest for the free TLR7 compound 1v270, and lower for both the Cationic PEG 1v270 Lip formulation and the Cationic 1v270 Lip formulation, supporting that formulation and delivery of 1v270 in cationic PEGylated liposomes is a method to reduce IL-6 secretion thereby reducing risks of toxicity associated with CRS (FIG. 3C). IL-10 which is secreted from a range of immune cells and is associated with suppressing immune responses is an important cytokine for immune homeostasis and is often associated with immune activation, with secretion after excessive immune responses. However, too high IL-10 levels will exert a cancer promoting activity, and is not beneficial for a potent anti-cancer immunotherapeutic approach. Our data demonstrated that by formulation of 1v270 into cationic liposomes we reduced IL-10 secretion 4 fold compared to the free 1v270 TLR7 agonist, supporting the use of cationic liposomes for a cancer immunotherapy approach (FIG. 3D).

For a prediction of risks of toxicity induction using the delivery approach, we compared secretion of toxicity associated IL-6 with levels of beneficial cytokines for an anti-cancer response IFNα and IL-12p70 as shown in FIGS. 3A-D. The Cationic PEG 1v270 Lip formulation showed at least 5-10 fold lower toxicity prediction compared to the free 1v270 TLR7 agonist, and approximately 2 fold lower toxicity prediction than the Cationic 1v270 Lip formulation. This supports use of the Cationic PEG 1v270 Lip formulation over the free compound as well as the non PEGylated Cationic 1v270 Lip formulation(FIG. 3E).

A potent cancer immunotherapy should preferably boost the immune system with anti-tumor cytokines IFNα and IL-12p70 with subsequent low IL-10 levels. As a measure to predict this effect we introduced an immunogenicity score, calculating the ratio between IFNα and IL-12p70 with the level of IL-10. The Cationic PEG 1v270 Lip formulation showed more than 20 fold better immunogenicity score compared to the free 1v270 TLR7 compound, and three fold better immunogenicity score compared to the Cationic 1v270 Lip formulation. This supports use of the Cationic PEG 1v270 Lip formulation over the free compound as well as the non-PEGylated Cationic 1v270 Lip formulation (FIG. 3F).

### Example 6: In Vivo Cytokine Secretion in Plasma of Mice Injected with Free 1v270 and Cationic Liposomes

In vivo analyses of cytokine responses stimulated by the free 1v270 compound, the Cationic PEG 1v270 Lip and Cationic 1v270 Lip formulations administered intravenously in mice and subsequent measurement of plasma cytokine levels at 2, 6 and 24 h after administration, showed that the Cationic PEG 1v270 Lip formulation was much more potent for induction of IFNα and IL-12p70 than both the free 1v270, but also the non-PEGylated Cationic 1v270 Lip formulation (FIG. 4A-D).

The cationic PEG 1v270 Lip was 12 fold more potent than the cationic 1v270 Lip formulation, and 20 fold more potent than the free 1v270 compound in induction of IL-12p70 (FIGS. 4A and 4B). The cationic PEG 1v270 Lip was 20 fold more potent than the cationic 1v270 Lip formulation in induction of IFNα, and the free 1v270 compound was unable to induce IFNα at all (FIGS. 4C and 4D). These data demonstrate that the cationic PEG 1v270 Lip formulation show superior in vivo anti-tumor cytokine production versus free 1v270 and cationic 1v270 Lip formulation without PEG. The cationic PEG 1v270 Lip formulation was also more potent for production of the proinflammatory cytokine IL-6, but only approximately 3 fold higher than the free 1v270 and the cationic 1v270 Lip formulation (FIGS. 4E and 4F). The cationic PEG 1v270 Lip formulation showed similar induction of the immunosuppressive cytokine IL-10 compared to free 1v270 and the cationic 1v270 Lip formulation (FIGS. 4G and 4H).

A similar toxicity prediction as performed in FIG. 3E was performed for the mouse data, and showed an 8 fold higher toxicity prediction based on the IL-6/IFNα ratio for the cationic 1v270 Lip compared to the cationic PEG 1v270 Lip (score for free 1v270 could not be determined because no IFNα was measured in mouse plasma at any timepoint). The free 1v270 showed an 8 fold higher toxicity prediction than the cationic PEG 1v270 Lip, which showed half the toxicity prediction compared to the cationic 1v270 Lip formulation (FIG. 41). Comparing immunogenicity scores in the same way as for FIG. 3F, the cationic PEG 1v270 Lip was at least 10 fold more potent than the non-PEGylated cationic 1v270 Lip formulation based on the IFNα/IL-10 ratio, and more than 2 fold more potent based on the IL-12p70/IL-10 ratio, and 20 fold more potent than free 1v270 treatment (FIG. 4J).

Taken together these mouse cytokine data demonstrates that the cationic PEG 1v270 Lip formulation is far more potent for induction of antitumor cytokines than both free 1v270 TLR7 agonist and the non-PEGylated cationic 1v270 Lip formulation. Although the cationic PEG 1v270 Lip formulation induced IL-6 to higher levels than free 1v270 and cationic 1v270 Lip, and similar IL-10 levels, the toxicity prediction and immunogenicity scores were in strong favor of using the cationic PEG 1v270 Lip formulation for a reduced risk of toxicity while retaining a strong immunogenic potential.

### Example 7: Tumor Growth Inhibition by Combined Oxaliplatin and Cationic PEG 1v270 Lip Treatment

A mouse tumor study with the syngenic colon cancer model CT26 was performed in Balb c mice (FIG. 5A). Mice were injected sc with the tumor cell line CT26 and allowed to grow until day 10 when the tumors were palpable. At this day mice were randomly allocated to groups with 8 mice per group, with an average tumor size of approximately 100 mm³. Mice were then injected iv. with the chemotherapy oxaliplatin formulated in long circulating liposomes at 8 mg/kg for three times with three days interval. Non-treated group served as control group for tumor growth without any anti-tumor treatment. Oxaliplatin is a strong inducer of immunogenic cell death, which makes tumor cells responsive to enhanced immune activation as seen for the Cationic PEG 1v270 Lip formulation. One control group received PBS and experienced fast tumor growth with mice sacrificed on approximately day 36 (FIG. 5A). Mice receiving oxaliplatin formulation alone showed a weak tumor growth delay, and one mouse experienced complete remission shown in the KaplanMeier plot on FIG. 5B. The combined oxaliplating formulation and Cationic PEG 1v270 Lip immunotherapy showed a significant tumor growth delay compared to the oxaliplatin formulation alone (p<0.025), demonstrating that the Cationic PEG 1v270 Lip formulation is indeed a strong type of cancer immunotherapy to combine with existing treatment like the chemotherapeutic drug Oxaliplatin. The combined group even showed complete remission in three out of the eight mice in this treatment group (FIG. 5B).

### Example 8: PEGylation of Cationic Liposomes Prevents Aggregation in Human Plasma

A clear advantage of using PEGylated liposomes for targeting immune cells in whole blood compared to non-PEGylated liposomes is the reduced tendency to aggregate of PEGylated liposomes compared to non-PEGylated liposomes. In order to investigate this we tested the Cationic TriArg PEG Lip and the Cationic TriArg Lip formulations by adding the liposome to human plasma that was prepared by centrifuging the blood at high speed (FIG. 6A). Pure plasma contains few events that might be exosomes, chylomicrons, VLDL particles ect. (low fluorescent signal) (panel A). In panel B, PEGylated liposomes are added. There is a small increase in the fluorescence of the events, but not in the size of the events meaning that the liposomes remain small and non-aggregated in human plasma. In panel C, the same experiment is made for non-PEGylated cationic liposomes. There is a very large increase in the number of events, as well as in both size and fluorescence intensity of the events, meaning the fluorescent liposomes aggregate to larger particles in human plasma, which is not desired for a formulation used for iv administration.

The data of FIG. 6A were quantified and shown in FIG. 6B, with all events that can be seen by flow cytometry (panel A), or only the fluorescent aggregates using the gating strategy from FIG. 6A (Panel B).

These data demonstrate that a strong advantage of using PEGylated liposomes for application to human whole blood is a reduced tendency to aggregate, and thereby risk of forming large aggregates in patients treated with this type of immunotherapy.

### Example 9: Targeting to Myeloid and Plasmacytoid Dendritic Cells in Human Whole Blood

Targeting to dendritic cell (DC) subsets in human whole blood was evaluated by performing the study as described in example 4, except larger blood volumes were used. Flow cytometry was performed using a similar procedure as described in example 4, except the DCs were gated using the following gating strategy (CD1c myeloid DCs:CD45⁺, HLA-DR⁺, CD1c, CD11c. CD11c myeloid DCs: CD45⁺, HLA-DR⁺, CD141, CD11c. plasmacytoidDCs: CD45⁺, CD11c⁻, CD303a, HLA-DR⁺). DC subsets positive for Topflour are shown in FIG. 7, where DCs positive for liposome uptake was shown between 60-nearly 100% of cells positive for liposome uptake. There was no clear difference between uptake for the three different subsets, but rather a difference for different donors. These data show that the Cationic PEG 1v270 Lip formulation targets all three DC subsets analyzed, when incubated in whole human blood. Untreated control group shows fluorescent signal in non-treated whole blood immune cells.

### Example 10: 5% 1v270 Content in Liposomes is the Most Potent Immune Inducer of Immune Response

To determine the optimal content of 1v270 in cationic PEGylated liposomes, liposomes were prepared with 0, 1, 2.5, 5 and 7.5% 1v270 content. Liposomes were prepared as the POPC:CHOL:DOTAP:1v270:DOPE-PEG2000 (35:30:25:5:5) formulation, where the amount of POPC and DOTAP was adjusted when changing the 1v270 content from 0 to 7.5% (Table 2). Formulations with 1v270 content above 7.5% did not form stable liposomes. The liposomes were incubated as described in example 5 and cytokine secretion of anti-tumor cytokines IL-12p70 and IFNα was measured. The 5% 1v270 formulation induced the highest level of cytokines, and significantly higher than with 1% and 2.5%, and a tendency to be more potent than the 7.5% formulation. Conclusively, the 5% 1v270 liposomes were the most potent for induction of IL-12p70 and IFNα cytokine (FIG. 8).

### Table 2:

Overview of cationic liposomes used to determine the optimal amount of the active TLR7 agonist compound 1v270. Liposome name, composition, molar ratio, surface charge (Zeta), size and Polydispersity Index (PDI) are shown. Liposomes were composed lipids as for Table I. Composition and molar ratio of each component in the liposomes is shown. Liposome surface charge is expressed in mV. Liposome size is measured in nanometer (nm). Error is expressed as SEM. Liposome names from Table II are used for example 10 and FIG. 8.

| Name | Composition | Molar ratio |
|---|---|---|
| 5% | POPC:Chol:DOTAP:DOPE-PEG2000:1v270 | 35:30:25:5:5 |
| 1v270 | | |
| 7.5% | POPC:Chol:DOTAP:DOPE-PEG2000:1v270 | 30:30:27.5:5:7.5 |
| 1v270 | | |
| 0% | POPC:Chol:DOTAP:DOPE-PEG2000:1v270 | 45:30:20:5:0 |
| 1v270 | | |
| 1% | POPC:Chol:DOTAP:DOPE-PEG2000:1v270 | 43:30:21:5:1 |
| 1v270 | | |
| 2.5% | POPC:Chol:DOTAP DOPE-PEG2000:1v270 | 30:30:22.5:5:2.5 |
| 1v270 | | |

| Name | Zeta (mV) | Size (nm) | PDI |
|---|---|---|---|
| 5% 1v270 | 19.91 ± 1.23 | 114.3 ± 1.7 | 0.055 |
| 7.5% 1v270 | 17.11 ± 1.56 | 124.6 ± 0.4 | 0.021 |
| 0% 1v270 | 26.50 ± 1.23 | 115.1 ± 2.3 | 0.063 |
| 1% 1v270 | 23.82 ± 1.43 | 116.5 ± 1.0 | 0.056 |
| 2.5% 1v270 | 23.66 ± 1.68 | 112.7 ± 2.6 | 0.073 |

### Example 11: Treatment of a Mammal with Cancer Using Immunostimulatory Cationic PEG 1v270 Lip Liposomes (Non-Antigen Specific)

To obtain an immune stimulatory liposome suitable for cancer treatment, a monocyte targeting liposome is prepared using e.g. POPC:CHOL:DOTAP:1v270:DOPE-PEG2000 (35:30:25:5:5). The compounds are mixed in organic solvent and dried to a lipid film. This film is hydrated in a buffer suitable for intravenous administration, e.g. containing saline and glucose. The Cationic PEG 1v270 Lip liposomes are administered intravenously to a cancer patient suffering from e.g. lung cancer, breast cancer, prostate cancer, leukemia, lymphoma or melanoma with e.g. a one-two week interval. Activated monocytes are known to migrate to peripheral tissue like the tumor environment, and are therefore suitable for enhancing the anti-tumor immune response in combination with currently approved cancer treatments that induce tumor cell death that makes the tumor cells visible to the activated immune cells. Combinations with clinically approved treatments with scientific indications for a benefit from combination with immunotherapy could be combination with radiotherapy to boost the abscopal effect in e.g. lung cancer patients, to combine with mAb therapy like Rituximab and Trastuzumab to boost the Antibody Dependent Cell Cytotoxicity (ADCC), to enhance responses towards immunogenic cell death induced by certain chemotherapy like doxorubicin, oxaliplatin, cyclophosphamide and mitoxantrone.

### Example 12: Treatment of a Mammal with Cancer Using Immune Stimulatory Cationic PEG 1v270 Lip Liposomes (Antigen Specific)

To obtain an antigen specific immune response liposomes are prepared as described in example 1+2, but with addition of an antigen peptide encoding whole or parts of the antigen of interest. The peptide antigen may be associated with a lipid anchor to ensure sufficient liposome association as seen for a 25 amino acid peptide sequence from the MUC1 tumor antigen, where a palmitoylated lysine residue ensures sufficient liposome association of the antigen (Sangha and Butts, Clin Cancer Res 2007; 13, 15 supp, 2007, 4652-54s). The antigen may be e.g. a MAGE antigen for treatment of melanoma, PSA for treatment of prostate cancer, a neoantigen or a third antigen or a combination of antigens. The antigen together with 1v270 are administered to a cancer patient corresponding to the loaded antigen. The liposomes are administered to the same patient for a number of times to boost an antigen specific response, preferably with 1-2 weeks interval.

### Example 13: Vaccine for Preventing Infectious Disease (e.g. Influenza Virus) Using Antigen Specific Immunostimulatory Cationic PEG 1v270 Lip Liposomes

To obtain an antigen specific immune response suitable for treatment of an infectious disease like e.g. influenza infections, hepatitis or third world virus infection liposomes are prepared using e.g. POPC:CHOL:DOTAP:1v270:DOPE-PEG2000 (35:30:25:5:5). Liposomes are prepared as in example 12, however, the antigen of interest is derived from whole or parts of proteins from the infectious agent, and modified with a similar anchor as outlined in example 10. The Cationic PEG 1v270 Lip liposome together with one or more infectious disease antigen(s) are administered to a human in a preventive or curative manner which may be with 2-3 weeks interval. This vaccine may be used for intravenous, intradermal, subcutaneous or intramuscular administration.

1. A cationic liposome comprising:
a) between 0-40 mol % cholesterol,
b) between 1-10 mol % PEG conjugated to a phospholipid,
c) at least one cationic lipid, and
d) at least one immunostimulating compound,
wherein the remaining components are phospholipids and wherein the zeta potential is in the range of 13-25 mV.

2. The cationic liposome according to claim 1, wherein the at least one immunostimulating compound is a ligand for an intracellular protein and/or receptor selected from the group consisting of TLR7, STING, TLR3, TLR8, TLR9, NOD1, NOD2, NOD5, NALP1, NALP2, NALP3, NALP12, NALP14, IPAF, NAIP, CIITA, RIG-I, MDA5, and LGP2.

3. The cationic liposome according to any of the preceding claims, wherein the cationic liposome preferentially adheres to monocytes and dendritic cells in fresh whole blood when compared to adherence to granulocytes, T-lymphocytes, B-lymphocytes and/or NK cells.

4. The cationic liposome according to any of the preceding claims, wherein the at least one immunostimulating agent is a TLR7 agonist, such as a TLR7 agonist selected from the group consisting of Formula (I), Formula (II), Formula (III) and Formula (IV). 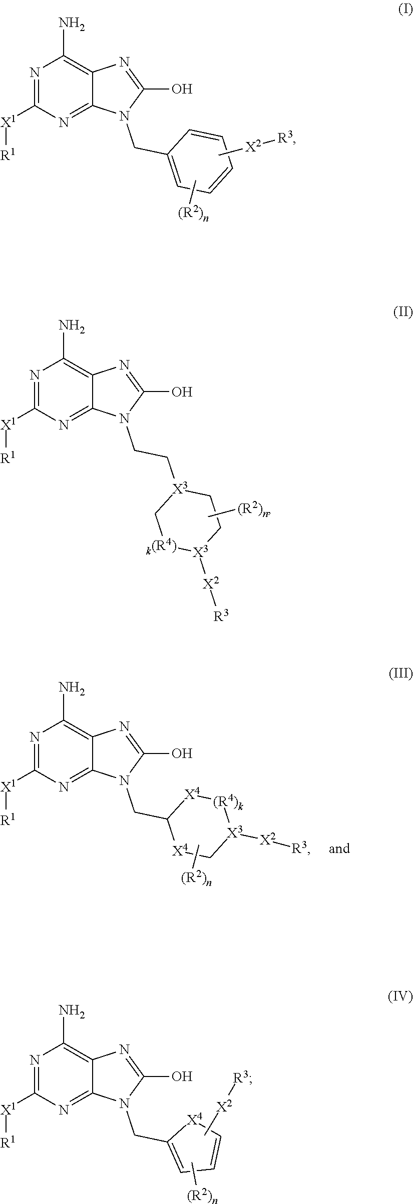
wherein X¹ is -0-, —S—, or —NR^{C};
R¹ is hydrogen, (C₁-C₁₀)alkyl, substituted (C₁-C₁₀)alkyl, C₆₋₁₀aryl, or substituted C₆₋₁₀aryl, C₅₋₉heterocyclic, substituted C₅₋₉heterocyclic;
R^{C} is hydrogen, C₁₋₁₀alkyl, or substituted C₁₋₁₀alkyl; or R^{C} and R¹ taken together with the nitrogen to which they are attached form a heterocyclic ring or a substituted heterocyclic ring;
each R² is independently —OH, (C₁-C₆)alkyl, substituted (C₁-C₆)alkyl, (C₁-C₆)alkoxy, substituted (C₁-C₆)alkoxy, —C(O)—(C₁-C₆)alkyl (alkanoyl), substituted —C(O)—(C₁-C₆)alkyl, —C(O)—(C₆-C₁₀)aryl (aroyl), substituted —C(O)—(C₆-C₁₀)aryl, —C(O)OH (carboxyl), —C(O)O(C₁-C₆)alkyl (alkoxycarbonyl), substituted —C(O)O(C₁-C₆)alkyl, —NR^{a}R^{b}, —C(O)NR^{a}R^{b} (carbamoyl), halo, nitro, or cyano, or R² is absent;
each R^{a} and R^{b} is independently hydrogen, (C₁-C₆)alkyl, substituted (C₁-C₆)alkyl, (C₃-C₈)cycloalkyl, substituted (C₃-C₈)cycloalkyl, (C₁-C₆)alkoxy, substituted (C₁-C₆)alkoxy, (C₁-C₆)alkanoyl, substituted (C₁-C₆)alkanoyl, aryl, aryl(C₁-C₆)alkyl, Het, Het (C₁-C₆)alkyl, or (C₁-C₆)alkoxycarbonyl;
wherein the substituents on any alkyl, aryl or heterocyclic groups are hydroxy, C₁₋₆alkyl, hydroxyC₁₋₆alkylene, C₁₋₆alkoxy, C₃₋₆cycloalkyl, C₁₋₆alkoxy C₁₋₆alkylene, amino, cyano, halo, or aryl;
n is 0, 1, 2, 3 or 4;
X² is a bond or a linking group; and
R³ is a phospholipid comprising one or two carboxylic esters;
X³ is —N— or —CH—;
R⁴ is —CH₂— or —CH(R²)—; and
k is 0 or 1;
X⁴ is —O—, —S—, —NH—, —N(R^{d})—, —CH₂—, or —CH(R²)—;
each R^{d} is independently —OH, (C₁-C₆)alkyl, substituted (C₁-C₆)alkyl, (C₁-C₆)alkoxy, substituted (C₁-C₆)alkoxy, —C(0)-(C₁-C₆)alkyl (alkanoyl), substituted —C(0)-(C₁-C₆)alkyl, —C(0)-(C₆-C₁₀)aryl (aroyl), substituted —C(O)—(C₆-C₀)aryl, —C(0)0(C₁-C₆)alkyl (alkoxycarbonyl), substituted —C(0)0(C₁-C₆)alkyl, —C(0)NR^{a}R^{b} (carbamoyl);
or a tautomer thereof;
or a pharmaceutically acceptable salt or solvate thereof, and
wherein the ring system of formula (II) is a piperidine ring with one heteroatom being an N atom and with the N-atom of the piperidine ring adjacent to X², and wherein the purine group in any of Formula (I), (II), (III), or (IV) is subject to tautomeric rearrangements.

5. The cationic liposome according to claim 4, wherein the TLR7 agonist has a structure according to Formula (IA).

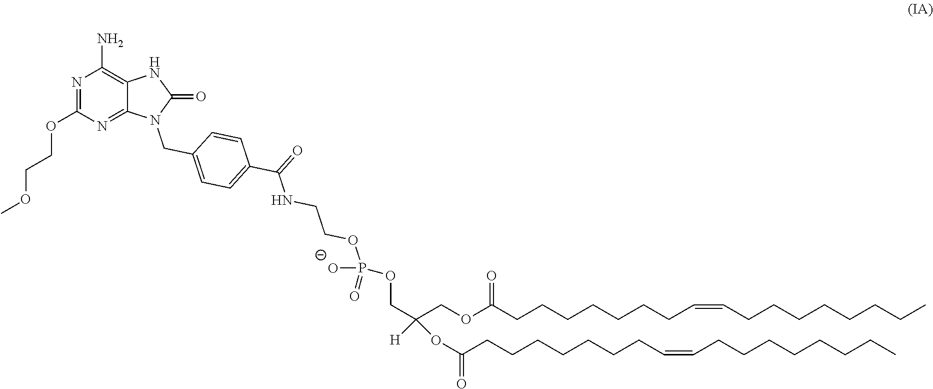 [SMILES: CCCCCCCC/C=C\CCCCCCCC(=O)OCC(COP(=O)([O-])OCCNC(=O)c1ccc(Cn2c(=O)[nH]c3c(N)nc(OCCOC)nc32)cc1)OC(=O)CCCCCCC/C=C\CCCCCCCC]

6. The cationic liposome according to any of the preceding claims wherein the content of cationic lipid in mol % multiplied by the charge of the cationic lipid is in the range of 16-30, such as 20-30, such as 20-25.

7. The cationic liposome according to any of the preceding claims, wherein the cationic lipid is selected from the group consisting of stearylamine (SA), lauryltrimethylammonium bromide; cetyltrimethyl-ammonium bromide, myristyl trimethylammonium bromide, dimethyldioctadecylammonium bromide (DDAB), 36-[N-(N′,N′-dimethylaminoethane)-carbamoyl]cholesterol (DC-Cholesterol), 1,2-ditetradecanoyl-3-trimethylammonium-propane (DMTAP), 1,2-dioctadecanoyl-3-trimethylammonium-propane (DOTAP) and DOTAP derivatives such as 1,2-di-(9Z-octadecenoyl)-3-trimethylammonium-propane and 1,2-dihexadecanoyl-3-trimethylammonium-propane, 1,2-di-(9Z-octadecenoyl)-3-dimethylammonium-propane (DODAP) and DODAP derivatives such as 1,2-ditetradecanoyl-3-dimethylammonium-propane, 1,2-dihexadecanoyl-3-dimethylammonium-propane, and 1,2-dioctadecanoyl-3-dimethylammonium-propane, 1,2-di-0-octadecenyl-3-trimethylammonium propane (DOTMA), 1,2-dioleoyl-c-(4′-trimethylammonium)-butanoyl-sn-glycerol (DOTB), dioctadecylamide-glycylspermine, SAINT-2, polycationic lipid 2,3-dioleyloxy-N-[2(spermine-carboxamido)ethyl]-N,N-dimethyl-1-propanaminiumtrifluoroacetate (DOSPA), 1-palmitoyl-2-oleoyl-sn-glycero-3-ethylphosphocholine (EPC) and GL67™, preferably the cationic lipid is DOTAP.

8. The cationic liposome according to any of the preceding claims, wherein the cationic lipid is 1,2-dioctadecanoyl-3-trimethylammonium-propane (DOTAP), preferably wherein the content of cationic lipid is in the range of 16-30 mol %, such as 20-30 mol %, such as 20-25 mol %.

9. The cationic liposome according to any of the preceding claims, wherein the content of the immunostimulating compound is in the range of about 0.1-50 mol %, for example about 2-40 mol %, for example about 5-30 mol %, for example about 10-20 mol %, for example about 2.5-7.5 mol %, such as about 3-7 mol %, preferably the content of the active ingredient is about 5 mol %.

10. A cationic liposome comprising:
a) between about 30-40 mol % POPC, such as about 35 mol % POPC,
b) between about 25-35 mol % cholesterol, such as about 30 mol % cholesterol,
c) between about 3-7 mol % DOPE-PEG2000, such as about 5 mol % DOPE-PEG2000,
d) between about 20-30 mol % DOTAP, such as about 25 mol % DOTAP, and
e) between about 2.5-7.5 mol % of the TLR7 agonist 1v270, such as about 5 mol % 1v270,
wherein the zeta potential is in the range of 13-25 mV.

11. The cationic liposome according to any of the preceding claims, wherein the cationic liposome comprises at least one immunostimulating compound and at least one further active ingredient.

12. The cationic liposome according to any of the preceding claims, wherein the cationic liposome comprises at least one immunostimulating compound and at least one antigen.

13. A pharmaceutical composition comprising the cationic liposome according to any one of claims 1-12.

14. The cationic liposome according to any one of claim 1-12 or the pharmaceutical composition according to claim 13, for use in prophylaxis, treatment or amelioration of cancer, an infectious disease, an inflammatory condition or disease, an autoimmune disease or allergy.

15. A lipid-based delivery system for targeting monocytes and dendritic cells in fresh whole blood, said system providing delivery to and release of at least one immunostimulating compound to the targeted monocyte, and said system comprising:
a) between 0-40 mol % cholesterol,
b) between 2-10 mol % PEG conjugated to a phospholipid,
c) at least one cationic lipid, and
d) at least one immunostimulating compound which is a ligand for an intracellular protein and/or receptor selected from the group consisting of TLR7, STING, TLR3, TLR8, TLR9, NOD1, NOD2, NOD5, NALP1, NALP2, NALP3, NALP12, NALP14, IPAF, NAIP, CIITA, RIG-I, MDA5, and LGP2;
wherein the remaining components are phospholipids and wherein the zeta potential is in the range of 13-25 mV,
said system allowing the formation of cationic liposomes, at which said cationic liposomes preferentially adheres to monocytes and dendritic cells in fresh whole blood when compared to adherence to granulocytes, T-lymphocytes, B-lymphocytes and/or NK cells.

16. A method for in vitro activation of monocytes and dendritic cells, comprising the steps of:
a) providing fresh blood,
b) administering a cationic liposome according to any one of claims 1-12 or the pharmaceutical composition according to claim 13 to said fresh blood, and
c) allowing said cationic liposome, lipid-based delivery system or pharmaceutical composition to react.

17. A method for in vivo activation of monocytes and dendritic cells in a subject, comprising administering the cationic liposome according to any one of claims 1-12 or the pharmaceutical composition according to claim 13 to said subject in an amount sufficient to activate or inhibit said monocytes and dendritic cells.

18. A method for ex vivo activation of monocytes and dendritic cells, comprising the steps:
a) administering the cationic liposome according to any one of claims 1-12 or the pharmaceutical composition according to claim 13 to a fresh blood sample obtained from a subject,
b) allowing said cationic liposome, lipid delivery system or pharmaceutical composition to react with said fresh blood sample.
